(19) United States
(12) Patent Application Publication
(10) Pub. No.: US 2004/0010033 A1
(43) Pub. Date: Jan. 15, 2004

(54) Non-peptide GnRH agents, methods and intermediates for their preparation

(75) Inventors: Mark Brian Anderson, Orinda, CA (US); Haresh N. Vazir, Salinas, CA (US); David Robert Luthin, Encinitas, CA (US); Genevieve DeGuzman Paderes, San Diego, CA (US); Ved P. Pathak, San Diego, CA (US); Lance Christopher Christie, Vista, CA (US); Yufeng Hong, San Diego, CA (US); Eileen Valenzuela Tompkins, Escondido, CA (US); Haitao Li, San Diego, CA (US); James Faust, Temecula, CA (US)

Correspondence Address: Keith D. Hutchinson, Agouron Pharmaceuticals, Inc., Legal Division, Patent Department, 10777 Science Center Drive, San Diego, CA, 92121, US

(73) Assignee: Pfizer Inc.

(21) Appl. No.: 10/353,160

(22) Filed: Jul. 8, 2003

Related U.S. Application Data
(62) Division of application No. 09/763,216, filed on Feb. 20, 2001, now abandoned.

(51) Int. Cl. A61K 31/34; C07D 307/02

(52) U.S. Cl. 514/471; 549/487

(57) ABSTRACT

Non-peptide GnRH agents capable of inhibiting the effect of gonadotropin-releasing hormone are described. Such compounds and their pharmaceutically acceptable salts, multimers, prodrugs, and active metabolites are suitable for treating mammalian reproductive disorders and steroid hormone-dependent tumors as well as for regulating fertility, where suppression of gonadotropin release is indicated. Methods for synthesizing the compounds and intermediates useful in their preparation are also described.

This application claims priority from and incorporates by reference in its entirety pending prior U.S. application Ser. No. 09/763,216 filed Feb. 20, 2001, which claims the benefit of U.S. Provisional Application Serial No. 60/097,520 filed Aug. 20, 1998.

### TECHNICAL FIELD AND INDUSTRIAL APPLICABILITY OF THE INVENTION

This invention relates generally to compounds that affect the action of human gonadotropin-releasing hormone (GnRH). More particularly, it relates to non-peptide GnRH antagonists or agonists and to their preparation. These non-peptide GnRH agents have advantageous physical, chemical and biological properties, and are useful medicaments for diseases or conditions mediated by modulation of the pituitary-gonadal axis. The compounds of the invention avoid the degradation and biodistribution problems of peptide agents.

### BACKGROUND OF THE INVENTION

Gonadotropin-Releasing Hormone (GnRH), also known as luteinizing hormone-releasing hormone (LH-RH), plays a central role in the biology of reproduction. A large variety of analogs have been used for an increasing number of clinical indications. The GnRH decapeptide (pyro-Glu-His-Trp-Ser-Tyr-Gly-Leu-Arg-Pro-Gly-NH₂ or p-EHWSYGLRPG-NH₂) is produced in neurons of the medial basal hypothalamus from a larger precursor by enzymatic processing. The decapeptide is released in a pulsatile manner into the pituitary portal circulation system where GnRH interacts with high-affinity receptors (7-Transmembrane G-Protein Coupled Receptors) in the anterior pituitary gland located at the base of the brain. In the pituitary, GnRH triggers the release of two gonadotropic hormones (gonadotropins): luteinizing hormone (LH) and follicle-stimulating hormone (FSH). In testes and ovaries, LH stimulates the production of testosterone and estradiol, respectively. FSH stimulates follicle growth in women and sperm formation in men. When correctly functioning, the pulse-timed release and concentration levels of GnRH are critical for the maintenance of gonadal steroidogenesis and for normal functions of reproduction related to growth and sexual development.

The pituitary response to GnRH varies greatly throughout life. GnRH and the gonadotropins first appear in the fetus at about ten weeks of gestation. The sensitivity to GnRH declines, after a brief rise during the first three months after birth, until the onset of puberty. Before puberty, the FSH response to GnRH is greater than that of LH. Once puberty begins, sensitivity to GnRH increases, and pulsatile LH secretion ensues. Later in puberty and throughout the reproductive years, pulsatile release of GnRH occurs throughout the day, with LH responsiveness being greater than that of FSH. Pulsatile GnRH release results in pulsatile LH and FSH release and hence testosterone and estradiol release from the gonads. After menopause, FSH and LH concentrations rise, and post-menopausal FSH levels are higher than those of LH.

Chronic administration of GnRH agonists and antagonists to animals or to man results in decreased circulating levels of both LH and FSH. GnRH agonists are compounds that mimic endogenous GnRH to stimulate receptors on the pituitary gland, resulting in release of LH and FSH. After a transient rise in gonadal hormone production or “flare” response, chronic administration of GnRH agonists results in a down-regulation of GnRH receptors. GnRH receptor down-regulation and desensitization of the pituitary results in a decrease of circulating levels of LH and FSH. In spite of the symptom-exacerbating hormonal flare experienced, GnRH agonists have been the treatment of choice for sex-steroid-dependent pathophysiologies. For example, GnRH agonists have been used to reduce testosterone production, thereby reducing prostate volume in benign prostatic hyperplasia (BPH) and slowing tumor growth in prostate cancer. These compounds have also been used to treat breast and ovarian cancers.

Recently, GnRH antagonists have become available for clinical evaluation. GnRH antagonists have an immediate effect on the pituitary without the observed flare associated with agonists. Use of GnRH antagonists (usually decapeptides) has been reported in the literature for treatment of breast, ovarian, and prostatic cancers. Other uses of antagonists, like agonists, include endometriosis (including endometriosis with pain), uterine myoma, ovarian and mammary cystic diseases (including polycystic ovarian disease), prostatic hypertrophy, amenorrhea (e.g., secondary amenorrhea), and precocious puberty. These compounds may also be useful in the symptomatic relief of premenstrual syndrome (PMS). Furthermore, antagonists may be useful to regulate the secretion of gonadotropins in male mammals to arrest spermatogenesis (e.g., as male contraceptives), and for treatment of male sex offenders. Importantly, GnRH antagonists (and agonists) have found utility in treatments where a reversible suppression of the pituitary-gonadal axis is desired.

The presence of GnRH receptors on anterior pituitary cells and several tumor cell types offers the opportunity to develop drugs that act upon these receptors to treat both hormone-dependent and hormone-independent cancers.

For over 50 years, androgen deprivation has been the most effective systematic therapy for the treatment of metastatic carcinoma of the prostate. The rationale is simple—the prostate gland requires androgens for proper growth, maintenance, and function. Yet, prostate cancer and benign prostate hyperplasia are common in men and develop in an environment of continuous androgen exposure. Thus, utilizing a GnRH antagonist to interrupt the pituitary-gonadal axis reduces androgen production and results in tumor growth modulation. Furthermore, GnRH antagonists may have a direct effect on tumor growth by blocking receptors on the tumor cells. For those cancer types that respond both to sex hormones and to GnRH directly, antagonists should be effective in slowing tumor growth by two mechanisms. Since GnRH receptors are present on many prostate and breast cancer cells, it has recently been speculated that GnRH antagonists may also be effective in treating non-hormone-dependent tumors. Recent literature examples indicate that GnRH receptors are present on a number of cancer cell lines, including:

Prostate Cancer: GnRH agonists exert both in vitro, and in vivo, a direct inhibitory action on the growth of both androgen-dependent (LNCaP) and androgen-independent (DU 145) human prostatic cancer cell lines. Montagnani et al, Arch. Ital. Urol. Androl. 1997, 69(4), 257-263. GnRH antagonist inhibit the growth of androgen-independent PC-3 prostate cancer in nude mice. Jungwirth et al., Prostate 1997, 32(3), 164-172.

Ovarian Cancer: The demonstration of GnRH receptors in human ovarian cancers provides a rationale for the use of therapeutic approaches based on GnRH analogues in this malignancy. Srkalovic et al., Int. J. Oncol. 1998, 12(3), 489-498.

Breast Cancer: Breast cancer is the most common type of cancer in women over the age of 40 and is the leading cause of cancer-related death in women. Systematic endocrine intervention represents a major treatment option for the management of advanced breast cancer, especially with estrogen-dependent cancers. The genes for gonadotropin-releasing hormone and its receptor are expressed in human breast with fibrocystic disease and cancer. Kottler et al., Int. J. Cancer 1997, 71(4), 595-599.

Heretofore, available GnRH antagonists have primarily been peptide analogs of GnRH. See, e.g., International Publication No. WO 93/03058. Peptide antagonists of peptide hormones are often quite potent; however, the use of peptide antagonists is typically associated with problems because peptides are degraded by physiological enzymes and often poorly distributed within the organism being treated. Thus, they have limited effectiveness as drugs. Consequently, there presently exists a need for non-peptide antagonists of the peptide hormone GnRH.

### SUMMARY OF THE INVENTION

An object of the invention is to develop small-molecule non-peptide GnRH antagonists that exploit both of the above-described mechanisms of action. Non-peptide GnRH agents have advantageous physical, chemical and biological properties compared to peptides, and will be useful medicaments for diseases mediated via the pituitary-gonadal axis and by directly targeting the receptor on tumor cells. There is a need to develop drugs that act upon these receptors to treat both hormone-dependent and hormone-independent cancers.

Another object of the invention is to provide non-peptide compounds that are GnRH agents (agonists or antagonists) that bind to GnRH receptors and thus modulate activity, especially those that are potent GnRH antagonists. Another object of the invention is to provide effective therapies for individuals needing therapeutic regulation of GnRH and to provide methods for treating diseases and conditions mediated by GnRH regulation.

Such objects have been achieved by the non-peptide GnRH compounds of the invention, which are useful as pharmaceuticals for indications mediated by GnRH regulation. The inventive compounds are pharmaceutically advantageous over peptide compounds since they provide better biodistribution and tolerance to degradation by physiological enzymes. The invention further provides methods of synthesizing the compounds as well as intermediate compounds useful for making the compounds.

The invention is directed to compounds of the general Formula I:

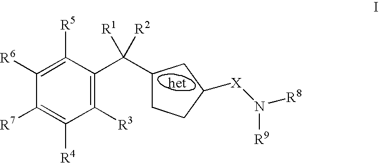

where:

X is selected from C═O, C═S, S═O, and S(O)₂;

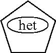

is a 5-membered heterocyclic ring containing from 1 to 4, preferably 2 or 3, heteroatoms selected from N, O, and S, wherein the ring may be saturated, partially unsaturated, or fully unsaturated, and may be aromatic;

R¹ and R² are independently selected from H and lower alkyl;

R³ is selected from H, halogen, and substituted and unsubstituted alkyl, alkenyl, alkynyl, cycloalkyl; heterocycle, aryl, heteroaryl, CH₂OR, OR, and C(O)OR, where R is selected from substituted and unsubstituted alkyl, alkenyl, alkynyl, cycloalkyl, heterocycle, aryl, and heteroaryl, and where the total number of carbon atoms present (not including any optional substituents) ranges from 1 to 12;

R⁴ and R⁵ are independently selected from H, halogen, and substituted and unsubstituted alkyl, alkenyl, alkynyl, cycloalkyl, heterocycle, aryl, heteroaryl, CH₂OR, OR, and C(O)OR, where R is as defined above; and where the total number of carbon atoms present (not including any optional substituents) ranges from 1 to 12;

R⁶ and R7 are independently selected from H, halogen, and substituted and unsubstituted alkyl, alkenyl, alkynyl, cycloalkyl, heterocycle, aryl, heteroaryl, CH₂OR, OR, and C(O)OR; where R is as defined above, and where the total number of carbon atoms present (not including any optional substituents) ranges from 1 to 12; or R⁶ and R⁷ taken together with the atoms to which they are bonded form an optionally substituted 5- or 6-membered ring optionally having up to four heteroatoms selected from O, N, and S;

R⁸ is a lipophilic moiety selected from substituted and unsubstituted alkyl, alkenyl, alkynyl, cycloalkyl, heterocycle, aryl, heteroaryl, CH₂OR, OR, and C(O)OR, where R is as defined above, and where the total number of carbon atoms present (not including any optional substituents) ranges from 6 to 20; and

R⁹ is selected from H and substituted and unsubstituted alkyl, preferably lower alkyl.

Preferred compounds of the invention are of the general formula II:

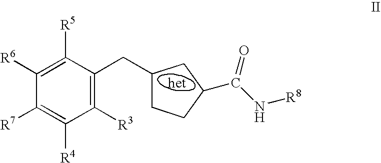

where the variables in the formula are as defined above.

Especially preferred compounds have the formula III:

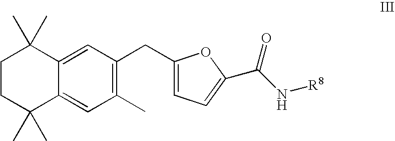

where R⁸ is defined above. Preferred R⁸ groups include: aryl, —CH₂-aryl, —CH₂-heteroaryl, —CH₂-cycloalkyl, and —(CH₂)ₙ—O-aryl where n is an integer of from 1 to 4.

Preferred compounds of the invention include:

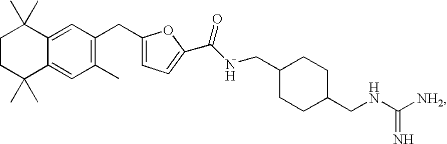

including both cis- and trans-isomers at the cyclohexyl substituent;

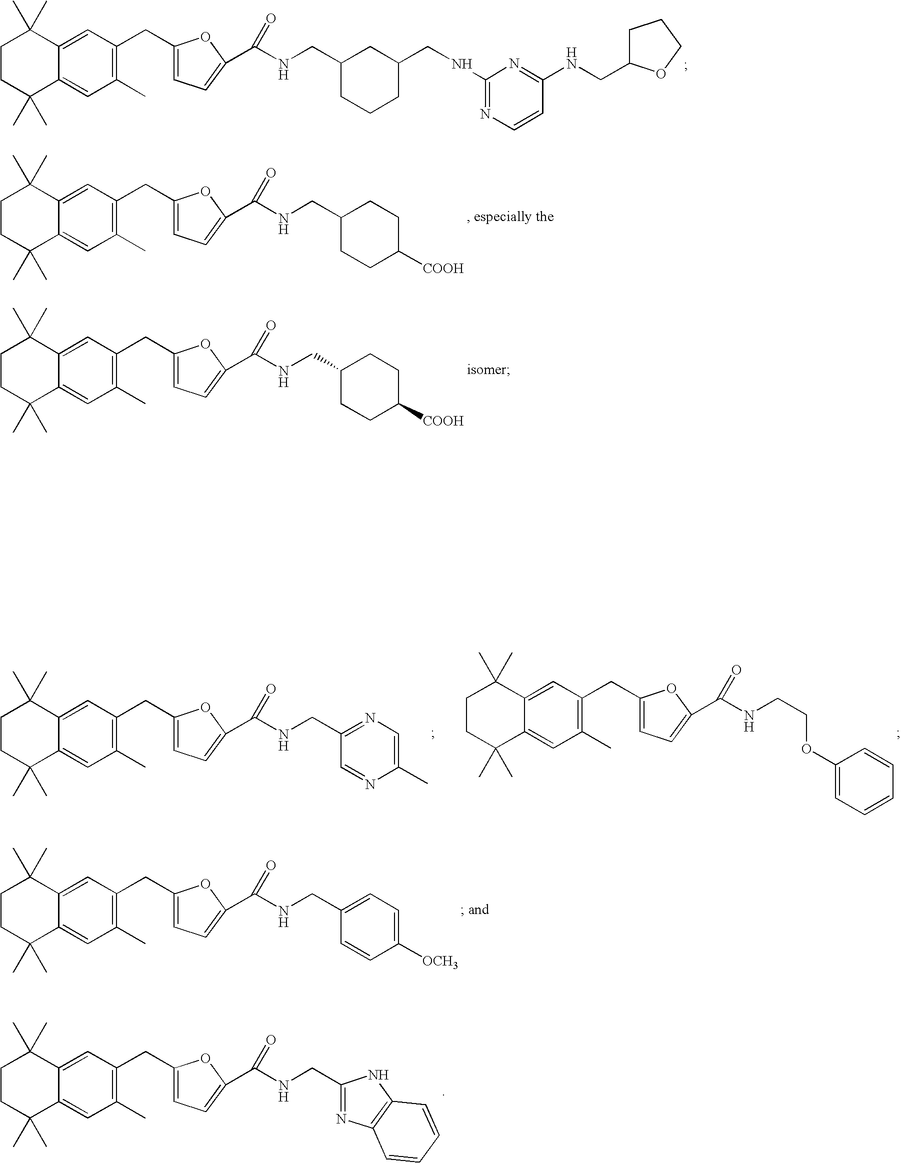

In addition to compounds of the above formulae, GnRH agents of the invention include pharmaceutically acceptable salts, multimeric forms, prodrugs, and active metabolites of such compounds. Such non-peptide agents are pharmaceutically advantageous over peptide agents since they provide better biodistribution and tolerance to degradation by physiological enzymes.

The invention also relates to pharmaceutical compositions comprising a therapeutically effective amount of a GnRH agent of the invention in combination with a pharmaceutically acceptable carrier or diluent. Moreover, the invention relates to methods for regulating the secretion of gonadotropins in mammals, comprising administering therapeutically effective amounts of GnRH agents of the invention.

The invention also relates to methods and intermediates useful for making compounds of the Formula I.

Other features, objects, and advantages of the invention will become apparent from the following detailed description of the invention and its preferred embodiments.

### DETAILED DESCRIPTION OF INVENTION AND PREFERRED EMBODIMENTS

Some of the compounds of the invention contain one or more centers of asymmetry, and may thus give rise to enantiomers, diastereoisomers, and other stereoisomeric forms. The invention is meant to include all such possible stereoisomers as well as their racemic and optically pure forms. When the compounds described herein contain olefinic double bonds, they are intended to encompass both E and Z geometric isomers.

The chemical formulae referred to herein may exhibit the phenomenon of tautomerism. As the structural formulae shown in this specification only depict one of the possible tautomeric forms, it should be understood that the invention nonetheless encompasses all tautomeric forms.

The term “alkyl” refers to straight- and branched-chain alkyl groups having one to twelve carbon atoms. Exemplary alkyl groups include methyl (Me), ethyl, n-propyl, isopropyl, butyl, isobutyl, sec-butyl, tert-butyl (tBu), pentyl, isopentyl, tert-pentyl, hexyl, isohexyl, and the like. The term “lower alkyl” designates an alkyl having from 1 to 8 carbon atoms (a C₁₋₈-alkyl). Suitable substituted alkyls include fluoromethyl, difluoromethyl, trifluoromethyl, 2-fluoroethyl, 3-fluoropropyl, hydroxymethyl, 2-hydroxyethyl, 3-hydroxypropyl, and the like.

The term “alkenyl” refers to straight- and branched-chain alkenyl groups having from 2 to 12 carbon atoms. Illustrative alkenyl groups include prop-2-enyl, but-2-enyl, but-3-enyl, 2-methylprop-2-enyl, hex-2-enyl, and the like.

The term “alkynyl” refers to straight- and branched-chain alkynyl groups having from 2 to 12 carbons atoms. Exemplary alkynyls include prop-2-ynyl, 3-methylpent-4-ynyl, hex-2-ynyl, and the like.

The term “carbocycle” refers to a monocyclic or polycyclic carbon ring structure (with no heteroatoms) having from 3 to 7 carbon atoms in each ring, which may be saturated, partially saturated, or unsaturated. Exemplary carbocycles include cycloalkyls and aryls.

The term “heterocycle” refers to a monocyclic or polycyclic ring structure with one or more heteroatoms selected from N, O, and S, and having from 3 to 7 atoms (carbon atoms plus any heteroatom(s)) in each ring, which may be saturated, partially saturated, or unsaturated. Exemplary heterocycles include tetrahydrofuranyl, tetrahydropyranyl, azetidinyl, pyrrolidinyl, piperidinyl, piperazinyl, and the like.

The term “cycloalkyls” as used herein refers to saturated carbocycles having 3 to 12 carbons, including bicyclic and tricyclic cycloalkyl structures. Suitable cycloalkyls include cyclopropyl, cyclobutyl, cyclopentyl, cyclohexyl, cycloheptyl, and the like.

The terms “aryls” and “heteroaryls” refer to monocyclic and polycyclic unsaturated or aromatic ring structures, with “aryl” referring to those that are carbocycles and “heteroaryl” referring to those that are heterocycles. Examples of aromatic ring structures include phenyl, naphthyl, 1,2,3,4-tetrahydronaphthyl, furyl, thienyl, pyrrolyl, pyridyl, pyridinyl, pyrazolyl, imidazolyl, pyrazinyl, pyridazinyl, 1,2,3-triazinyl, 1,2,4-oxadiazolyl, 1,3,4-oxadiazolyl, 1-H-tetrazol-5-yl, indolyl, quinolinyl, benzofuranyl, benzothiophenyl (thianaphthenyl), and the like. Such moieties may be optionally substituted by one or more suitable substituents, for example, a substituent selected from a halogen (F, Cl, Br or I); lower alkyl; OH; NO₂; CN; CO₂H; O-lower alkyl; aryl; aryl-lower alkyl; CO₂CH₃; CONH₂; OCH₂CONH₂; NH₂; SO₂NH₂; OCHF₂; CF₃; OCF₃; and the like. Such moieties may also be optionally substituted by a fused-ring structure or bridge, for example OCH₂—O.

The term “aryl-lower alkyl” means a lower alkyl bearing an aryl. Examples include benzyl, phenethyl, pyridylmethyl, naphthylmethyl, and the like. The aryl-lower alkyl may be optionally substituted.

In general, the various moieties or functional groups for variables in Formula I may be optionally substituted by one or more suitable substituents. Exemplary substituents include a halogen (F, CI, Br, or I), lower alkyl, —OH, —NO₂, —CN, —CO₂H, —O-lower alkyl, -aryl, -aryl-lower alkyl, —CO₂CH₃, —CONH₂, —OCH₂CONH₂, —NH₂, —SO₂NH₂, haloalkyl (e.g., —CF₃, —CH₂CF₃), —O-haloalkyl (e.g., —OCF₃, —OCHF₂), and the like.

In addition to compounds of the Formula I, GnRH agents of the invention include pharmaceutically acceptable salts, multimeric forms, prodrugs, and active metabolites of compounds of the Formula I. Such non-peptide agents are pharmaceutically advantageous over peptide agents since they provide better biodistribution and tolerance to degradation by physiological enzymes.

Additionally, Formula I is intended to cover, where applicable, solvated as well as unsolvated forms of the compounds. Thus, Formula I includes compounds having the indicated structure, including the hydrated as well as the non-hydrated forms.

As indicated above, GnRH agents in accordance with the invention also include active tautomeric and stereoisomeric forms of the compounds of the Formula I, which may be readily obtained using techniques known in the art. For example, optically active (R) and (S) isomers may be prepared via a stereospecific synthesis, e.g., using chiral synthons and chiral reagents, or racemic mixtures may be resolved using conventional techniques.

GnRH agents further include multivalent or multimeric forms of active forms of the compounds of the Formula I. Such “multimers” may be made by linking or placing multiple copies of an active compound in close proximity to each other, e.g., using a scaffolding provided by a carrier moiety. Multimers of various dimensions (i.e., bearing varying numbers of copies of an active compound) may be tested to arrive at a multimer of optimum size with respect to receptor binding. Provision of such multivalent forms of active receptor-binding compounds with optimal spacing between the receptor-binding moieties may enhance receptor binding (see, for example, Lee et al., Biochem., 1984, 23:4255). The artisan may control the multivalency and spacing by selection of a suitable carrier moiety or linker units. Useful moieties include molecular supports containing a multiplicity of functional groups that can be reacted with functional groups associated with the active compounds of the invention. A variety of carrier moieties may be used to build highly active multimers, including proteins such as BSA (bovine serum albumin) or HAS, peptides such as pentapeptides, decapeptides, pentadecapeptides, and the like, as well as non-biological compounds selected for their beneficial effects on absorbability, transport, and persistence within the target organism. Functional groups on the carrier moiety, such as amino, sulfhydryl, hydroxyl, and alkylamino groups, may be selected to obtain stable linkages to the compounds of the invention, optimal spacing between the immobilized compounds, and optimal biological properties.

Additionally, GnRH agents of the invention include pharmaceutically acceptable salts of compounds of the Formula I. The term “pharmaceutically acceptable” refers to salt forms that are pharmacologically acceptable and substantially non-toxic to the subject being administered the GnRH agent. Pharmaceutically acceptable salts include conventional acid-addition salts or base-addition salts formed from suitable non-toxic organic or inorganic acids or inorganic bases. Exemplary acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Exemplary base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from organic bases such as amines, benzylamines, piperidines, and pyrrolidines.

The term “prodrug” refers to a metabolic precursor of a compound of the Formula I (or a salt thereof) that is pharmaceutically acceptable. A prodrug may be inactive when administered to a subject but is converted in vivo to an active compound of the Formula I. The term “active metabolite” refers to a metabolic product of a compound of the Formula I that is pharmaceutically acceptable and effective. Prodrugs and active metabolites of compounds of the Formula I may be determined using techniques known in the art.

A variety of known assays and techniques may be employed to determine the level of activity of various forms of the compounds in the GnRH system. Ligand-binding assays are used to determine interaction with the receptor of interest. Where binding is of interest, a labeled receptor may be used, where the label is a fluorescer, enzyme, radioisotope, or the like, which registers a quantifiable change upon the binding of the receptor. Alternatively, the artisan may provide for an antibody to the receptor, where the antibody is labeled, which may allow for amplification of the signal. Binding may also be determined by competitive displacement of a ligand bound to the receptor, where the ligand is labeled with a detectable label. Where agonist and/or antagonist activity is of interest, an intact organism or cell may be studied, and the change in an organismic or cellular function in response to the binding of the compound of interest may be measured. Various devices are available for detecting cellular response, such as a microphysiometer available from Molecular-Devices, Redwood City, Calif. In vitro and in vivo assays useful in measuring GnRH antagonist activity are known in the art. See, e.g., Bowers et al., “LH suppression in cultured rat pituitary cells treated with 1 ng of LHRH,” Endocrinology, 1980, 106:675-683 (in vitro,) and Corbin et al., “Antiovulatory activity (AOA) in rats,” Endocr. Res. Commun. 1975, 2:1-23 (in vivo). Particular test protocols that may be used are described below.

For example, GnRH-receptor antagonists may be functionally assessed by measurement of change in extracellular acidification rates as follows. The ability of compounds to block the extracellular rate of acidification mediated by GnRH in HEK 293 cells expressing human GnRH receptors is determined as a measure of the compound's antagonist activity in vitro. Approximately 100,000 cells/chamber are immobilized in agarose suspension medium (Molecular Devices) and perfused with unbuffered MEM media utilizing the Cytosensor® Microphysiometer (Molecular Devices). Cells are allowed to equilibrate until the basal acidification rate remains stable (approximately one hour). Control dose-response curves are performed to GnRH (10⁻¹¹M to 10⁻⁷M). Compounds are allowed to incubate 15 minutes prior to stimulation with GnRH, and are assessed for antagonist activity. After incubation with test compounds, repeat dose-response curves to GnRH in the presence or absence of various concentrations of the test compounds are obtained. Schild regression analysis is performed on compounds to determine whether compounds antagonize GnRH-mediated increases in extracellular acidification rates through a competitive interaction with the GnRH receptor.

In another test, accumulation of total inositol phosphates may be measured by formic acid extraction from cells, followed by separation of the phosphates on Dowex columns. Cells are split using trypsin into two 12-well plates and pre-labeled with ³H-myoinositol (0.5 Ci-2 mCi per mL) for 16-18 hours in inositol-free medium. The medium is then aspirated and the cells rinsed with either 1X HBSS, 20 mM HEPES (pH 7.5), or serum-free DMEM, 1X HBSS, 20 mM HEPES (pH 7.5) containing agonist, and 20 mM LiCl is then added and the cells are incubated for the desired time. The medium is aspirated and the reaction stopped by addition of ice-cold 10 mM formic acid, which also serves to extract cellular lipids. Inositol phosphates are separated by ion-exchange chromatography on Dowex columns, which are then washed with 5 mL of 10 mM myoinositol and 10 mM formic acid. The columns are then washed with 10 mL of 60 mM sodium formate and 5 mM borax, and total inositol phosphates are eluted with 4.5 mL 1M ammonium formate, 0.1M formic acid.

Preferred GnRH agents of the invention include those having a Kᵢ value of about 10 μM or less. Especially preferred GnRH agents are those having a Kᵢ value in the nanomolar range.

Preferred compounds of the inventions are shown in the following table:

| | STRUCTURAL | |
|---|---|---|
| COMPOUND NO. | FORMULA | mol. weight |
| 9 | 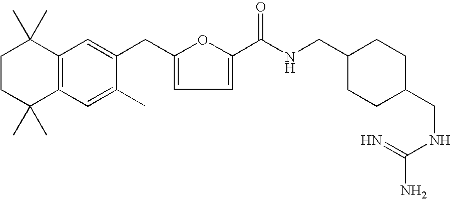 | 492.704 |
| 10 | 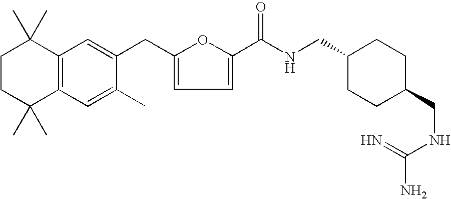 | 492.704 |
| 11 | 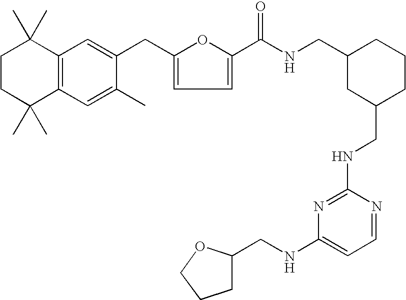 | 627.869 |
| 12 | 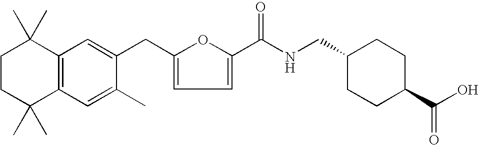 | 465.63 |
| 13 | 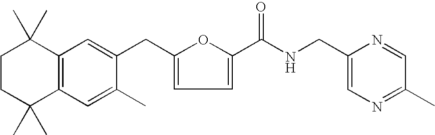 | 431.577 |
| 14 | 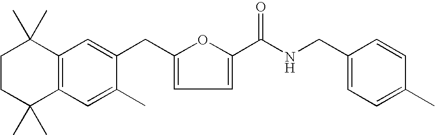 | 429.6 |
| 15 | 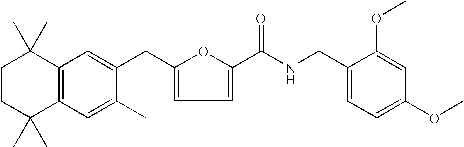 | 475.625 |
| 16 | 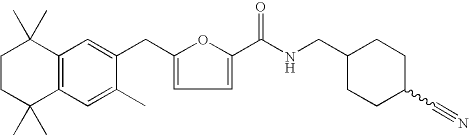 | 446.631 |
| 17 | 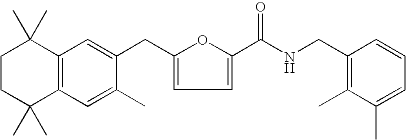 | 443.627 |
| 18 | 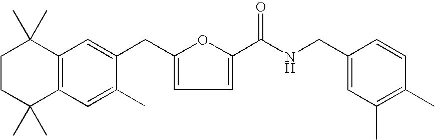 | 443.627 |
| 19 | 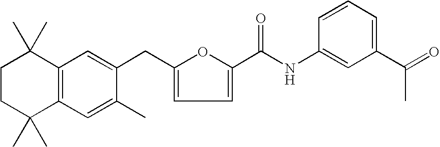 | 443.584 |
| 20 | 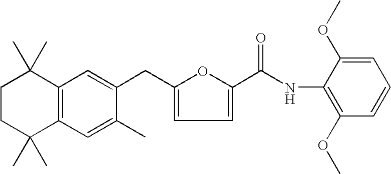 | 461.599 |

Pharmaceutical compositions according to the invention comprise an effective GnRH-suppressing amount of at least one GnRH agent according to the invention and an inert or pharmaceutically acceptable carrier or diluent. These compositions may be prepared in a unit-dosage form appropriate for the desired mode of administration, e.g., parenteral or oral.

To treat diseases or conditions mediated by GnRH agonism or antagonism, a pharmaceutical composition of the invention is administered in a suitable formulation prepared by combining a therapeutically effective amount (i.e., a GnRH-modulating amount effective to achieve therapeutic efficacy) of at least one GnRH agent of the invention (as an active ingredient) with one or more pharmaceutically suitable carriers or diluents. Such formulations may be prepared according to conventional procedures, e.g., by appropriately mixing, granulating, and compressing or dissolving the ingredients in known manners. Optionally, one or more different active ingredients, such as different GnRH antagonists, may be employed in a pharmaceutical composition.

The pharmaceutical carrier may be either a solid or liquid. Exemplary solid carriers include lactose, sucrose, talc, gelatin, agar, pectin, acacia, magnesium stearate, stearic acid, and the like. Illustrative of liquid carriers are syrup, peanut oil, olive oil, water, and the like. Similarly, the carrier or diluent may include time-delay or time-release materials known in the art, such as glyceryl monostearate or glyceryl distearate, alone or in combination with a wax, ethylcellulose, hydroxypropylmethylcellulose, methylmethacrylate, or the like.

A variety of pharmaceutical forms can be employed. For example, if a solid carrier is used, the preparation may be in the form of a tablet, hard-gelatin capsule, powder, pellet, troche, or lozenge. The amount of solid carrier may vary widely, with an exemplary amount ranging from about 25 mg to about 1 g. If a liquid carrier is used, the preparation may be in the form of a syrup, emulsion, soft-gelatin capsule, sterile injectable solution, suspension in an ampoule or vial, or non-aqueous liquid suspension.

To obtain a stable, water-soluble dosage form, a pharmaceutically acceptable salt of a compound of Formula I may be dissolved in an aqueous solution of an organic or inorganic acid, such as 0.3M solution of succinic acid or, more preferably, citric acid. If a soluble salt form is not available, the agent may be dissolved in one or more suitable cosolvents. Examples of suitable cosolvents include alcohol, propylene glycol, polyethylene glycol 300, polysorbate 80, gylcerin, and the like in concentrations ranging from 0% to 60% of the total volume. In an exemplary embodiment, a compound of Formula I is dissolved in DMSO and diluted with water. The composition may also be in the form of a solution of a salt form of a compound of the Formula I in an appropriate aqueous vehicle, such as water, or isotonic saline or dextrose solutions.

The pharmaceutical compositions of the present invention may be manufactured using conventional techniques, e.g., mixing, dissolving, granulating, dragee-making, levigating, emulsifying, encapsulating, entrapping, or lyophilizing processes. Pharmaceutical compositions may be formulated in a conventional manner using one or more physiologically acceptable carriers comprising excipients or auxiliaries selected to facilitate processing of the active compounds into pharmaceutical preparations. An appropriate formulation is selected in view of the route of administration chosen.

For preparing injectable preparations, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation and may be selected from those known in the art.

For oral administration, the agents may be formulated readily by combining the active ingredient(s) with pharmaceutically acceptable carriers known in the art. Such carriers enable the compounds of the invention to be formulated as tablets, pills, dragees, capsules, liquids, gels, syrups, slurries, suspensions, and the like, for oral ingestion by a patient to be treated. Pharmaceutical preparations for oral use can be obtained by combining one or more agents with a solid excipient, optionally grinding the resulting mixture into granules, and processing the mixture of granules after adding suitable auxiliaries, if desired, to obtain tablets or dragee cores. Suitable excipients include fillers such as sugars (e.g., lactose, sucrose, mannitol, or sorbitol) and cellulose preparations (e.g., maize starch, wheat starch, rice starch, potato starch, gelatin, gum, methyl cellulose, hydroxypropylmethylcellulose, sodium carboxymethylcellulose, and/or polyvinylpyrrolidone (PVP)). If desired, disintegrating agents may be added, such as cross-linked PVP, agar, or alginic acid or a salt thereof such as sodium alginate.

Dragee cores are provided with suitable coatings. For this purpose, concentrated sugar solutions may be used, which may optionally contain gum arabic, PVP, Carbopol™ gel, polyethylene glycol, titanium dioxide, lacquer solutions, and/or one or more suitable organic solvents. Dyestuffs or pigments may be added to the tablets or dragee coatings for identification or to characterize different combinations of active compound doses.

Pharmaceutical forms that are suitable for oral administration include push-fit capsules made of gelatin, as well as soft, sealed capsules made of gelatin and a plasticizer, such as glycerol or sorbitol. The push-fit capsules may contain the active ingredient(s) in admixture with one or more fillers such as lactose, binders such as starches, and/or lubricants such as talc or magnesium stearate, and, optionally, stabilizers. In soft capsules, the active compound may be dissolved or suspended in a suitable liquid, such as fatty oil, liquid paraffin, or liquid polyethylene glycol. In addition, stabilizers may be added. For buccal administration, the compositions may take the form of tablets or lozenges formulated in a conventional manner.

For administration by inhalation, the compounds for use according to the present invention may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide, or another suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the agent and a suitable powder base such as lactose or starch.

The agents may be formulated for parenteral administration by injection, e.g., by bolus injection or continuous infusion. Formulations for injection may be prepared in unit-dosage form, e.g., in ampoules, or in multi-dose containers, with an added preservative. The compositions may take such forms as suspensions, solutions, or emulsions in oily or aqueous vehicles, and may contain formulatory agents such as suspending, stabilizing, and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters such as ethyl oleate or triglycerides, or liposomes. Aqueous injectable suspensions may contain substances increasing the viscosity of the suspension, such as sodium carboxymethyl cellulose, sorbitol, or dextran. Optionally, the suspension may also contain suitable stabilizers or agents increasing the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated as rectal compositions, such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system (VPD is a solution of 3% w/v benzyl alcohol, 8% w/v of the nonpolar surfactant polysorbate 80, and 65% w/v polyethylene glycol 300, made up to volume in absolute ethanol). The VPD co-solvent system (VPD:5W) is comprised of VPD diluted 1:1 with a 5% dextrose-in-water solution. This co-solvent system dissolves hydrophobic compounds well, and the resulting formulation produces low toxicity upon systemic administration. As will be apparent, the proportions of a suitable co-solvent system may be varied in light of the solubility and toxicity characteristics. Furthermore, the identity of the co-solvent components may be varied: for example, other low-toxicity nonpolar surfactants may be used instead of polysorbate 80; the fraction size of polyethylene glycol may be varied; one or more other biocompatible polymers (e.g., PVP) may be added or replace polyethylene glycol; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs and may be used to formulate suitable preparations. Certain organic solvents such as dimethylsulfoxide also may be employed, although this may cause an increase in toxicity. Additionally, delivery may be achieved using a sustained-release system, such as semipermeable matrices of solid hydrophobic polymers containing the therapeutic agent. Various sustained-release materials are available and known to those skilled in the art. Sustained-release capsules may, depending on their chemical nature, release the compounds for a period lasting from a few weeks or up to over 100 days. Depending on the chemical nature and the biological stability of the therapeutic agent, additional techniques for protein stabilization may be readily employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter-ions. Pharmaceutically acceptable salts may be formed with many acids, including hydrochloric, sulfuric, acetic, lactic, tartaric, malic, succinic, and like acids. Salts tend to be more soluble in aqueous or other protonic solvents than are the corresponding free-base forms.

It will be appreciated that the actual dosages of the agents used in the compositions of this invention will vary according to the particular complex being used, the particular composition formulated, the mode of administration, and the particular site, host, and disease being treated. Optimal dosages for a given set of conditions may be ascertained by those skilled in the art using conventional dosage-determination tests in view of the experimental data for a given compound. For oral administration, an exemplary daily dose generally employed will be from about 0.001 to about 1000 mg/kg of body weight, with courses of treatment repeated at appropriate intervals. Administration of prodrugs may be dosed at weight levels that are chemically equivalent to the weight levels of the fully active compounds.

Examples of specific pharmaceutical preparations in accordance with the invention are provided below.

Parenteral Composition: To prepare a pharmaceutical composition of this invention suitable for administration by injection, 100 mg of a pharmaceutically acceptable water-soluble salt of a compound of Formula I is dissolved in DMSO and then mixed with 10 mL of 0.9% sterile saline. The resulting mixture is incorporated into a unit-dosage form suitable for administration by injection.

Oral Composition: To prepare an orally administerable pharmaceutical composition, 100 mg of a compound of Formula I is mixed with 750 mg of lactose. The resulting mixture is incorporated into a unit-dosage form suitable for oral administration, such as a hard-gelatin capsule.

### SYNTHESIS OF GnRH REAGENTS AND COMPOUNDS

A. Building Block Example:

Naphthalene-Based Building Blocks: A useful acylating agent is prepared by sequential Friedel-Crafts alkylations and is shown below:

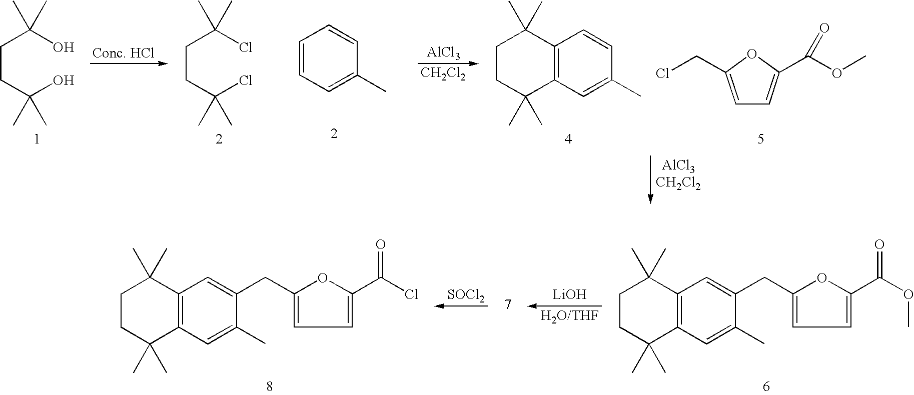

1,1,4,4,6-pentamethyl-1,2,3,4-tetrahydronaphtalene 4: To a solution of 2,5 dichloro-2,5 dimethylhexane 2 (10 g, 54.7 mmol) in toluene (270 Ml, 0.2 M) is slowly added aluminum trichloride (5.47 g, 41 mmol) as a solid over a 15-minute period. The reaction is complete after 10 minutes as assayed by tlc in hexanes. The unreacted aluminum trichloride is quenched slowly with water over 10 minutes. Additional toluene (250 mL) is added to extract the product from the aqueous layer. The organic layer is passed through a pad of silica gel (40 g) and eluted with toluene. The organic layer is evaporated in vacuo to dryness to yield 1,1,4,4,6-pentamethyl-1,2,3,4-tetrahydronaphtalene 4 (11 g, 97% yield). NMR 1.29 (s, 6H), 1.28 (s, 6H), 1.69 (s, 4H), 2.32 (s, 3H), 7.22 (d, 1H), 7.12 (s, 1H), 6.97 (dd, 1H).

Methyl 5-[(3,5,5,8,8-pentamethyl-5,6,7,8-tetrahydro-2-naphthalenyl)methyl]-2-furoate 6: To a solution containing 1,1,4,4,6-pentamethyl-1,2,3,4-tetrahydronaphtalene 4 (20 g, 99 mmol) and methyl 5-(chloromethyl)-2-furoate 5 (17.28 g, 99 mmol) in methylene chloride (500 mL, 0.2 M), aluminum trichloride (16.46 g, 124 mmol) is added slowly as a solid at the reflux temperature of methylene chloride. The solution is refluxed for an additional two hours. The reaction is monitored by tlc in 10% ethyl acetate/hexanes solution. The reaction is cooled to room temperature and the unreacted aluminum trichloride is quenched with water over 15 minutes. The crude product is extracted with methylene chloride and passed through silica gel (80 g) and eluted with methylene chloride. The solvent is evaporated in vacuo to syrup. The crude product us purified with silica gel (300 g) via a plug filtration column. Methyl 5-[(3,5,5,8,8-pentamethyl-5,6,7,8-tetrahydro-2-naphthalenyl)methyl]-2-furoate 6 is eluted with 2% ethyl acetate/hexanes to afford 15.4 g (46% yield). NMR 1.25 (s, 6H), 1.28 (s, 6H), 1.67 (s, 4H), 2.23 (s, 3H), 3.89 (s,3H), 3.97 (s, 2H), 5.95 (d, 1H), 7.09 (m, 3H).

5-[(3,5,5,8,8-pentamethyl-5,6,7,8-tetrahydro-2-naphthalenyl)methyl]-2-furoic acid 7: To a solution containing methyl 5-[(3,5,5,8,8-pentamethyl-5,6,7,8-tetrahydro-2-naphthalenyl) methyl]-2-furoate 6 (15.1 g, 44 mmol) in MeOH (175 mL) and water (175 mL), a solution of NaOH (3.53 g, 88.3 mmol) in water (29 mL) is added. The reaction mixture is stirred overnight. After completion as judged by tlc, the solution is acidified with 1M HCl to pH 2. The crude product is extracted into organic layer using ethyl acetate, and concentrated to afford 5-[(3,5,5,8,8-pentamethyl-5,6,7,8-tetrahydro-2-naphthalenyl)methyl]-2-furoic acid 7 (15.0 g, 99% yield). NMR 1.26 (s, 6H), 1.28 (s, 6H), 1.68 (s, 4H), 2.24 (s, 3H), 4.00 (s, 2H), 6.01 (d, 1H), 7.10 (s, 2H), 7.23 (d, 1H).

5-[(3,5,5,8,8-pentamethyl-5,6,7,8-tetrahydro-2-naphthalenyl)methyl]-2-furoyl chloride 8: To a solution containing 5-[(3,5,5,8,8-pentamethyl-5,6,7,8-tetrahydro-2-naphthalenyl)methyl]-2-furoic acid 7 (20.15 g, 61.77 mmol) in methylene chloride (310 mL), thionyl chloride (45 mL, 617 mmol) is added. The reaction is refluxed for 5 hours and another batch of thionyl chloride (45 mL, 617 mmol) is added. The reaction is stirred overnight at room temperature. The solution is concentrated to a syrup and passed through a pad of silica gel (50 g), washed with 3% hexanes, and concentrated in vacuo to afford 5-[(3,5,5,8,8-pentamethyl-5,6,7,8-tetrahydro-2-naphthalenyl)methyl]-2-furoyl chloride 8 (17 g, 80% yield). NMR 1.26 (s, 6H), 1.28 (s, 6H), 1.68 (s, 4H), 2.25 (s, 3H), 4.00 (s,2H), 6.11 (d,1H), 7.10 (s, 1H), 7.11 (s, 1H) , 7.41 (d, 1H).

Additional building blocks can be prepared under these reaction conditions which contain a variety of functional groups contained in the general formula shown above.

B. Acylation Examples:

The next scheme shows several examples which can use the general synthetic procedure for acylations given below.

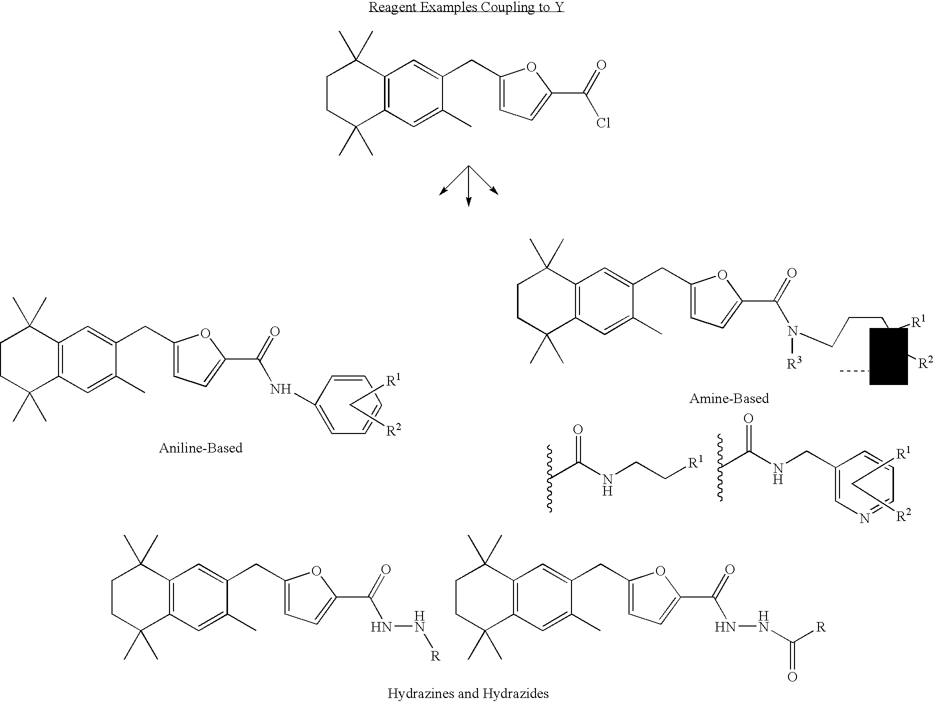

Amines are dissolved or suspended in dichloromethane, dichloroethane, ethyl acetate, acetonitrile, or the like (0.2M concentration) followed by the addition of the acid chloride reagent (1.00 mmol. equiv.). To the mixture is added triethyl amine (5.00 mmol. equiv.) and the reaction stirred at room temperature for 12-48 hours. The solvents are removed in vacuo. The product is purified by column chromatography on silica gel and eluted with an appropriate elution solvent (e.g., 3:1 hexanes:ethyl acetate). The solvents are removed in vacuo to yield the acylated product.

As an alternative, the reaction mixture is diluted with dichloromethane (five times the amount of dichloromethane used) and washed with saturated sodium bicarbonate. The organic layer is dried over magnesium sulfate and filtered. The product is purified by column chromatography on silica gel and eluted with an appropriate elution solvent (e.g., 3:1 hexanes:ethyl acetate). The solvents are removed in vacuo to yield the acylated product.

Using the general reaction protocol, large numbers of compounds can be readily prepared and assayed for their activities either as pure or impure materials. The reaction protocol works on anilines, amines, benzyl amines, hydrazines, hydrazides, alcohols and the like.

Specific examples showing a variety of structures acylated according to a general procedure are shown below:

| COMPOUND NO. | STRUCTURE | mol. weight |
|---|---|---|
| 9 | 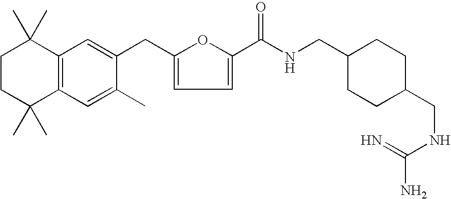 | 492.704 |
| 10 | 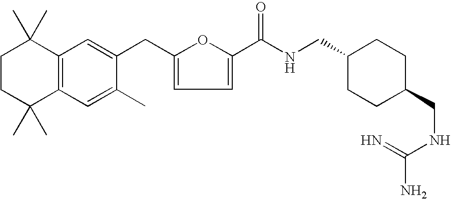 | 492.704 |
| 11 | 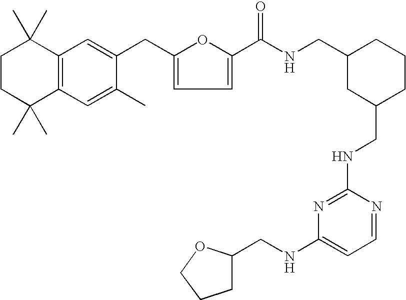 | 627.869 |
| 12 | 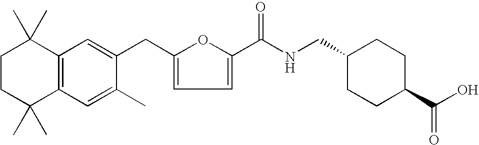 | 465.63 |
| 13 | 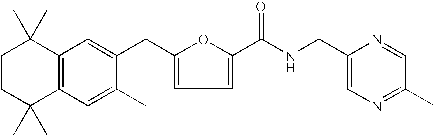 | 431.577 |
| 14 | 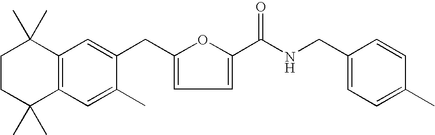 | 429.6 |
| 15 | 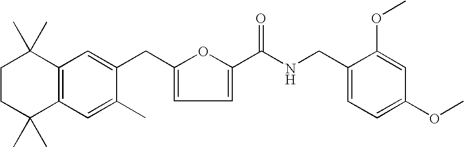 | 475.625 |
| 16 | 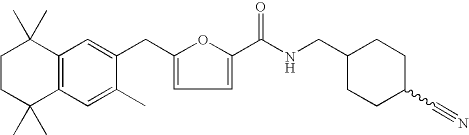 | 446.631 |
| 17 | 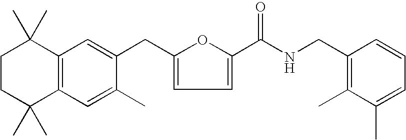 | 443.627 |
| 18 | 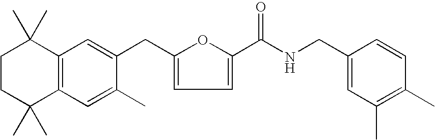 | 443.627 |
| 19 | 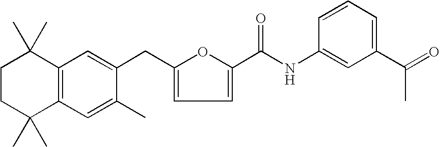 | 443.584 |
| 20 | 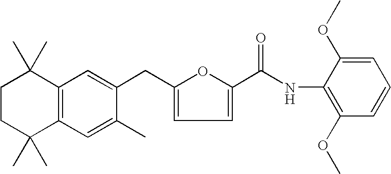 | 461.599 |

C. Synthesis and Acylation of Guanidine-Containing Compounds:

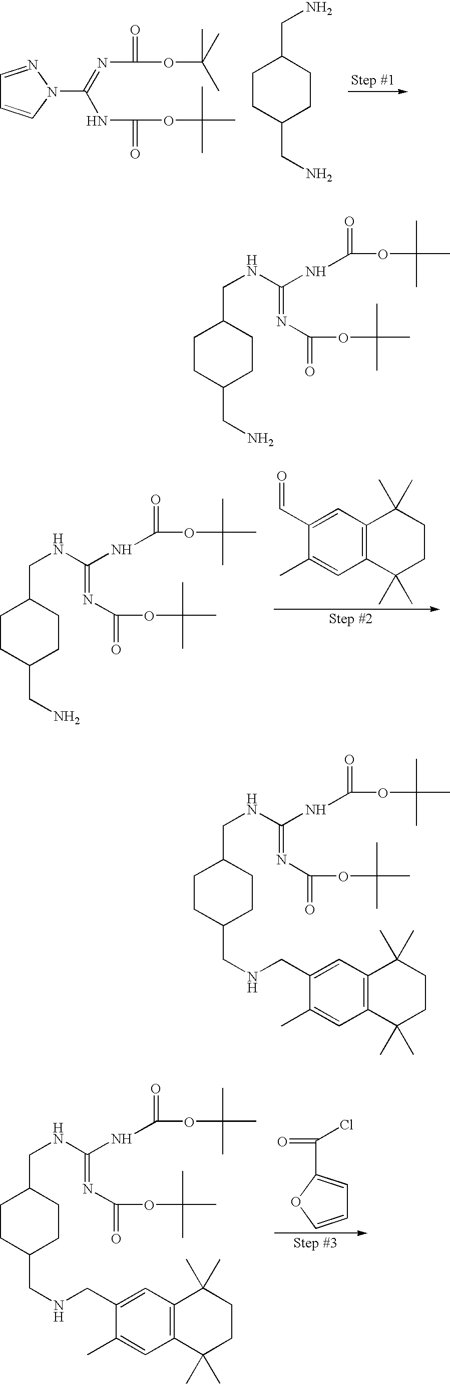

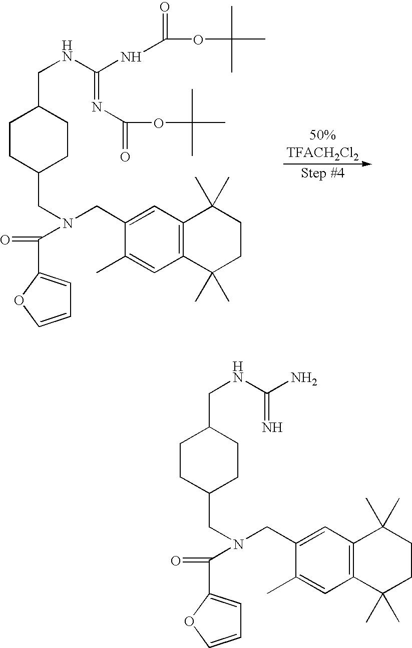

Step 1—Preparation of Protected Compound by 1-(N,N′-diBoc)-guanidinomethylation: Alternative Steps 1(A) and 1(B) below provide two general 1-(N,N′-diBoc)-guanidinomethylation procedures.

Step 1(A): To a solution of diamine (2.00 mmol equiv.) in THF (0.7 M) is added a solution of 1-H-pyrazole-1-(N,N-bis(tert-butoxycarbonyl)carboxamidine) (1.00 mmol equiv.) in THF (0.7M). The solution is stirred at room temperature for 3 hours (h), or until no further transformation can be observed by tlc (thin-layer chromatography). The solvent is removed under reduced pressure to give a syrupy residue, which is taken up in ethyl acetate (˜1.5 times the volume amount of THF used in the reaction or the volume of solvent needed to dissolve the amount of residue obtained) and washed with water until neutral pH. The organic layer is washed with brine, dried over MgSO₄, and concentrated. The product is purified by column chromatography on silica gel and eluted with an appropriate elution solvent (which may be readily determined, e.g., using 5% MeOH in dichloromethane as a starting point). The solvents are removed in vacuo to afford the 1-(N,N′-diBoc)-guanidinomethyl-linked-amine. In addition other reagents can be used to place a protected N,N′-diBoc-guanidine unit on diamines, such as 1,3-bis(tert-butoxycarbonyl)-2-methyl-2-thiopseudourea (CAS No. 107819-90-0). Alternatively, the 1-H-pyrazole-1-(N,N-bis(tert-butoxycarbonyl)carboxamidine) can be added directly as a solid, rather than as a solution as described above.

Step 1(B): To a solution of diamine (1.00 mmol equiv.) in THF (0.07M) is added portionwise as a solid (over a 10-minute time period) 1-H-pyrazole-1-(N,N-bis(tert-butoxy-carbonyl)carboxamidine) (1.00 mmol equiv.). The solution is stirred at room temperature for 0.5 hour. The solvent is removed under reduced pressure to give a syrupy residue, which is taken up in ethyl acetate (0.5 times the volume amount of THF used in the reaction, or the volume of solvent needed to dissolve the amount of residue obtained) and washed twice with water. The layers are separated, and the product is purified by column chromatography on silica gel and eluted with 100% ethyl acetate to remove any non-polar impurities and then with 100% isopropyl alcohol to give the pure product. The solvents are removed in vacuo to afford the desired product. Typical TLC conditions are 15:85:0.1 methanol/chloroform/acetic acid. Typical yields range from 40% to 44% of the desired protected compound.

Step 2—Reductive Amination (optional): Reductive amination may be accomplished in a suitable manner. For reductive amination of aldehydes and ketones with sodium triacetoxyborohydride, see generally: Abdel-Magid et al., J. Org. Chem., 1996, 61:3849. Two alternate reductive-aminations procedures are described below.

Step 2(A): 3,5,5,8,8-Pentamethyl-5,6,7,8-tetrahydro-2-naphth-aldehyde (1.00 mmol equiv.) and 1-(N,N′-diBoc)-guanidinomethyl-linked-amine (1.00 mmol equiv.) are dissolved in methanol (0.09M). Then, 1% glacial acetic acid in methanol solution (10% of the volume of methanol used) is added followed by NaCNBH₃ (1.00 mmol equiv.), and the reaction contents are stirred overnight. The reaction is assayed by TLC to reveal three components (aldehyde, desired product, and starting guanidine derivative). The reaction is terminated by adding water (50% of the volume of methanol used), extracted with dichloromethane (10 times the volume of methanol used), and washed with saturated sodium bicarbonate. The organic layer is dried over magnesium sulfate, filtered, and concentrated. The product is purified by column chromatography on silica gel and eluted with an appropriate elution solvent (e.g., 3:1 ethyl acetate in hexanes to remove the unreacted aldehyde, followed by elution with 1:1 ethyl acetate in hexanes), obtaining the desired reductive amination product. In some cases, warming to reflux for 2 hours will facilitate the imine formation reaction. See also, Abdel-Magid et al., J. Org. Chem., 1996, 61:3849, which describes the reductive amination of aldehydes and ketones with sodium triacetoxyborohydride.

Step 2(B): 3,5,5,8,8-Pentamethyl-5,6,7,8-tetrahydro-2-naphth-aldehyde (1.00 mmol equiv.) and 1-(N,N′-diBoc)-guanidinomethyl-linked-amine (1.00 mmol equiv.) are dissolved in methanol (0.09M). Then, NaBH₄ (1.00 mmol equiv.) is added (in ethanol via the additional small-scale procedures given below, or carefully as a solid) and the reaction contents are stirred overnight. The reaction is assayed by TLC to reveal three components (aldehyde, desired product and starting guanidine derivative). The reaction is terminated by the addition of water (50% of the volume of methanol used), extracted with dichloromethane (10 times the volume of methanol used), and washed with saturated sodium bicarbonate. The organic layer is dried over magnesium sulfate, filtered, and concentrated. The product is purified by column chromatography on silica gel and eluted with an appropriate elution solvent (as can be readily determined by the skilled artisan or, for example, with 3:1 ethyl acetate in hexanes to remove the unreacted aldehyde followed by elution with 1:1 ethyl acetate in hexanes) to obtain the desired reductive-amination product. In some cases, warming to reflux for 2 hours should facilitate the imine-formation reaction.

Step 3—Acylation: The products from the reductive amination (1.00 mmol equiv.) are dissolved in dichloromethane (˜0.2 to 0.05M, depending on solubilities of the substrates), followed by the addition of triethylamine (2.00 mmol equiv.) and 2-furoyl chloride reagent 8 (1.00 mmol equiv.). The reaction contents are stirred overnight at room temperature (RT). The reaction mixture is diluted with dichloromethane (5 times the amount of dichloromethane used) and washed with saturated sodium bicarbonate. The organic layer is dried over magnesium sulfate and filtered. The product is purified by column chromatography on silica gel and eluted with an appropriate elution solvent (e.g., 3:1 hexanes:ethyl acetate). The solvents are removed in vacuo to yield the acylated product.

Step 4—Basic Group Deprotection: The product from the acylation step (1.00 mmol equiv.) is dissolved in a solution of 25-50% TFA in dichloromethane (0.02M), and the reaction contents are stirred at room temperature (15-20 minutes; solution becomes slight reddish-orange). The reaction contents are stirred for an additional 1 hour and 20 minutes or until the BOC deprotection is complete. The reaction is terminated by concentration in vacuo, followed by the addition of water/acetonitrile (0.006M) and lyophilization overnight. The final compound is purified by high-performance liquid chromatography (HPLC) methodology. The solvents are removed in vacuo (yields range from 30% to 50%) to give the product.

An alternate procedure for removing of N, N′-bis-BOC guanidines using tin tetrachloride, which can give the corresponding guanidinium chloride salts, is described in Miel et al., Tetrahedron Letters, 1997, 38:7865-7866.

Compound 9 may be prepared according to the steps shown above with the exclusion of step #2, as shown in the following scheme:

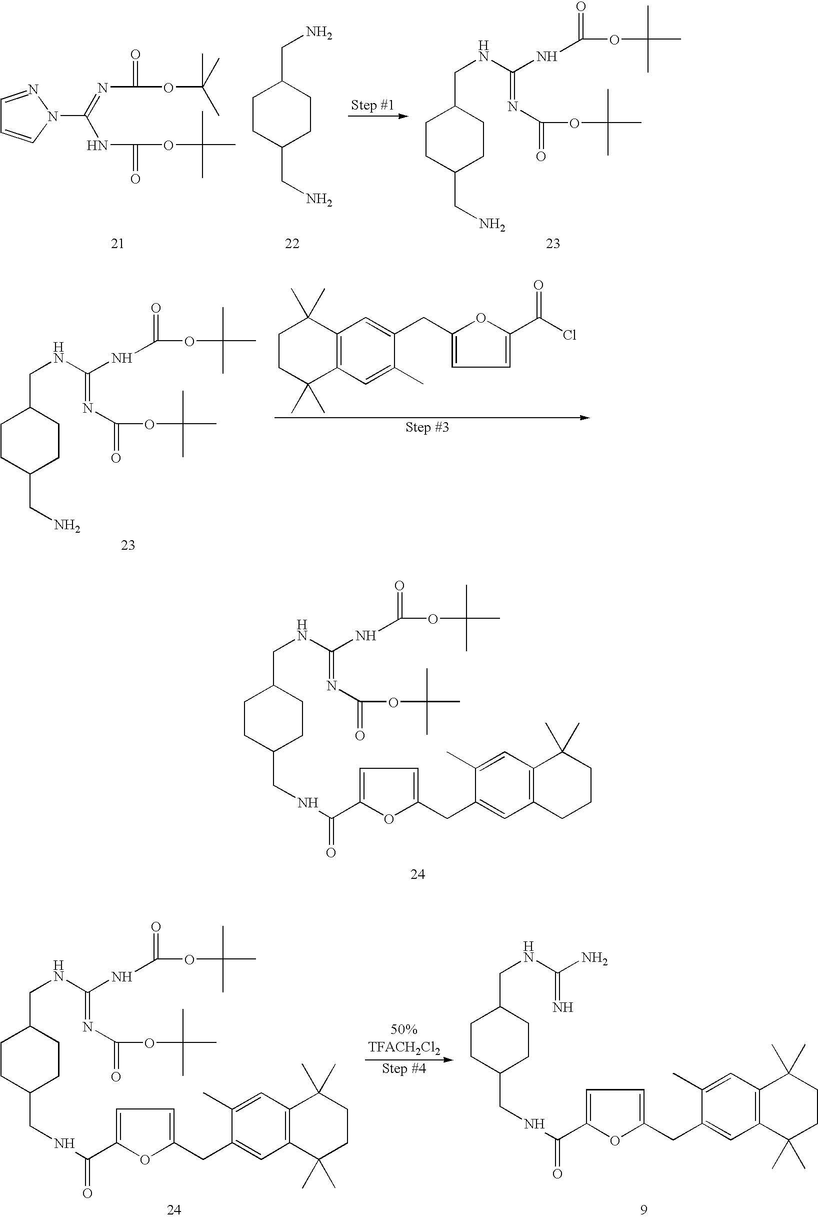

Preparation of Reagents: Reagents useful for synthesizing compounds may be obtained or prepared according to techniques known in the art. For example, the preparation of free amines from common salt forms and stock reagent solutions can be useful for small-scale reactions. See also Abdel-Magid et al., “Reductive amination of aldehydes and ketones with sodium triacetoxyborohydride,” J. Org. Chem., 1996, 61:3849.

Methanolic solutions of the free bases can be prepared from hydrochloride, dihydrochloride, hydrobromide, or other salts when the free base is soluble in methanol. In this procedure, once the sodium methoxide is added, care should be taken to prevent exposure to air, since amine free bases, particularly primary amines, absorb carbon dioxide from the air to form salts. A 10-mL quantity of a 0.1M solution of a free base in methanol may be prepared as follows. Weigh 1.0 mmol of a monohydrochloride salt into a tared Erlenmeyer flask containing a stirring bar, and add 7 mL of methanol. To the stirred slurry, add 229 mL (1.0 mmol, 1 equiv.) of sodium methoxide in methanol (25 wt %, 4.37M), stopper the flask, and stir the mixture vigorously for 2 hours. The slurry will sometimes change in appearance as a finer, milky precipitate of sodium chloride is formed. Filter the slurry through a 15-mL medium fritted glass funnel, wash the filter case with 1-2 mL methanol, transfer the filtrate to a 20-mL vial, and dilute to 10 mL with methanol. The theoretical yield of sodium chloride is nearly 59 mg, but the recovery is usually not quantitative, owing to a slight solubility in methanol. For a dihydrochloride salt, a second equivalent of sodium methoxide is required (458 mL).

A 0.5M solution of sodium borohydride in ethanol may be prepared as follows. Sodium borohydride (520 mg, 13.8 mmol) is stirred in pure (non-denatured) anhydrous ethanol (25 mL) for ˜2-3 minutes. The suspension is filtered through a medium fritted glass funnel to remove a small amount of undissolved solid (typically about 5% of the total mass of borohydride, or 25 mg). The filtrate should appears as a colorless solution that evolves only a little hydrogen. This solution should be used immediately, as it decomposes significantly over a period of a few hours, resulting in the formation of a gelatinous precipitate. Sodium borohydride is hygroscopic, so avoid exposure to air by making the solution at once after weighing the solid. Sodium borohydride has a solubility of about 4% in ethanol at room temperature. This corresponds to a little over 0.8M. However, sometimes a small percentage of the solid remains undissolved regardless of the concentration being prepared, even after stirring for ≧5 minutes.

To perform small-scale synthesis of compounds of the Formula I, the reactions described below may be performed to prepare various reactants useful in the reaction scheme described above. As with the rest of the specification, all temperatures in the following description are in degrees Celsius and all parts and percentages are by weight, unless indicated otherwise.

Various starting materials and other reagents may be purchased from commercial suppliers, such as Aldrich Chemical Company or Lancaster Synthesis Ltd., and used without further purification, unless otherwise indicated. Tetrahydrofuran (THF) and N,N-dimethylformamide (DMF) are purchased from Aldrich in SureSeal® bottles and used as received. All solvents are purified by using standard methods in the art, unless otherwise indicated.

The reactions set forth below are performed under a positive pressure of nitrogen or with a drying tube, at ambient temperature (unless otherwise stated), in anhydrous solvents, and the reaction flasks are fitted with rubber septa for the introduction of substrates and reagents via syringe. Glassware is oven-dried and/or heat-dried. Analytical thin-layer chromatography is performed on glass-backed silica gel 60° F. 254 plates (Analtech (0.25 mm)) and eluted with the appropriate solvent ratios (v/v). The reactions are assayed by TLC and terminated as judged by the consumption of starting material.

The tip plates are visualized with a p-anisaldehyde spray reagent or phosphomolybdic acid reagent (Aldrich Chemical, 20 wt % in ethanol) and activated with heat. Work-ups are typically done by doubling the reaction volume with the reaction solvent or extraction solvent and then washing with the indicated aqueous solutions using 25% by volume of the extraction volume (unless otherwise indicated). Product solutions are dried over anhydrous Na₂SO₄ prior to filtration, and evaporation of the solvents is under reduced pressure on a rotary evaporator and noted as solvents removed in vacuo. Flash column chromatography (Still et al., A. J. Org. Chem., 1978, 43:2923) is conducted using Baker-grade flash silica gel (47-61 mm) and a silica gel:crude material ratio of about 20:1 to 50:1, unless otherwise stated. Hydrogenolysis is done at the pressure indicated or at ambient pressure.

¹H-NMR spectra are recorded on a Bruker instrument operating at 300 MHz, and ¹³C-NMR spectra are recorded operating at 75 MHz. NMR spectra are obtained as CDCl₃ solutions (reported in ppm), using chloroform as the reference standard (7.25 ppm and 77.00 ppm) or CD₃OD (3.4 and 4.8 ppm and 49.3 ppm), or an internal tetramethylsilane standard (0.00 ppm) when appropriate. Other NMR solvents are used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Infrared spectra are recorded on a Perkin-Elmer FT-IR Spectrometer as neat oils, as KBr pellets, or as CDCl₃ solutions, and when reported are in wave numbers (cm⁻¹). The mass spectra are obtained using LSIMS or electrospray. All melting points are uncorrected.

Preparation of the Building Block 1-H-pyrazole-1-carboxamidine:

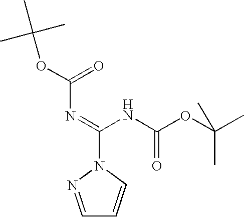

1-H-pyrazole-1-carboxamidine is prepared according to Bernatowicz et al., J. Org. Chem., 1992, 57:2497-2502 (and references therein), and protected with di-tert-butyldicarbonate to give 1-H-pyrazole-1-(N,N-bis(tert-butoxycarbonyl)carboxamidine) according to Drake et al., Synth., 1994, 579-582.

Preparation of 1-(N,N′-diBoc)-guanidinomethyl-4-aminomethylcyclohexane:

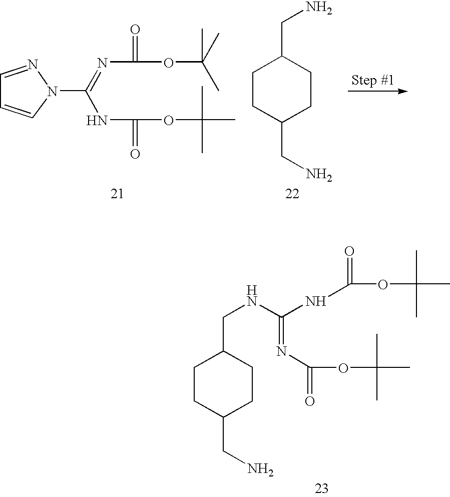

To a solution of 1,4-bis-aminomethyl-cyclohexane 22 (20 g, 0.14 mol) in THF (200 mL) is added a solution of 1-H-pyrazole-1-(N,N-bis(tert-butoxycarbonyl)carboxamidine) 21 (22.0 g, 0.07 mol) in THF (100 mL). (Note that 1-H-pyrazole-1-(N,N-bis(tert-butoxycarbonyl) carboxamidine) does not need to be dissolved in THF; rather it may be added neat as a solid to the process.) The solution is stirred at room temperature for 3 hours. The solvent is removed under reduced pressure to give a syrupy residue, which is taken up in ethyl acetate (500 mL) and washed with water until neutral pH. The organic layer is washed with brine, dried over MgSO₄, and concentrated. The product is purified by column chromatography on silica gel and eluted with 5% MeOH in dichloromethane. The solvents are removed in vacuo to afford 11.6 g (43% yield) of 1-(N,N′-diBoc)-guanidinomethyl-4-aminomethyl cyclohexane (Compound 23). ¹H NMR (CDCl₃) δ 11.5 (br s, 1H), 8.35 (br s, 1H), 3.26 (dt, 2H), 2.52 (dd, 2H), 1.82-0.97 (m, 28H, with singlet at 1.5).

An alternate preparation of 1-(N,N′-diBoc)-guanidinomethyl-3-aminomethylcyclohexane is as follows. To a solution of cis/trans 1,4-bis-aminomethyl-cyclohexane (9.0 g, 63.3 mmol) in THF (903 mL, 0.07M) is added portionwise as a solid (over a 10-minute period) 1-H-Pyrazole-1-(N,N-bis(tert-butoxycarbonyl)carboxamidine) (19.6 g, 63.3 mmol). The solution is stirred at room temperature for 0.5 hour. The solvent is removed under reduced pressure to give a syrupy residue, which is taken up in ethyl acetate (500 mL) and washed twice with water. The layers are separated and the product is purified by column chromatography on silica gel and eluted with 100% ethyl acetate to remove any non-polar impurities, followed by elution with 100% isopropyl alcohol, to give the pure product. The solvents are removed in vacuo to afford 10.2 g (42% yield) of 1-(N,N′-diBoc)-guanidinomethyl-4-aminomethylcyclohexane. ¹H NMR (CDCl₃) δ 11.5 (br s, 1H), 8.35 (br s, 1H), 3.26 (dt, 2H), 2.52 (dd, 2H), 1.82-0.97 (m, 28H, with singlet at 1.5).

Reductive Amination:

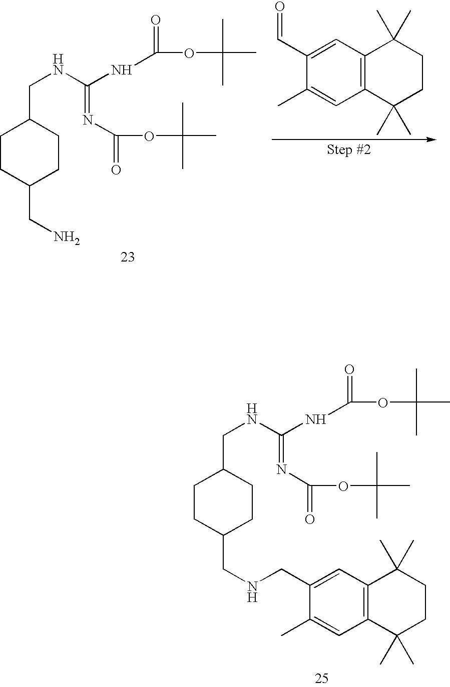

3,5,5,8,8-Pentamethyl-5,6,7,8-tetrahydro-2-naphth-aldehyde (0.2021 g, 0.88 mmol) and 1-(N,N′-diBoc)-guanidinomethyl-4-aminomethylcyclohexane (Compound 23, 0.337 g, 0.88 mmol) are dissolved in methanol (10 mL). Then, 1% glacial acetic acid in methanol (100 μL) solution is added followed by NaCNBH₃ (55.4 mg, 0.88 mmol, 1.0 equiv.), and the reaction contents are stirred overnight. The reaction is assayed by TLC to reveal three components (aldehyde, desired product, and starting guanidine derivative). The reaction is terminated by the addition of water (˜5 mL), extracted with dichloromethane (˜100 mL), and washed with saturated sodium bicarbonate. The organic layer is dried over magnesium sulfate, filtered, concentrated, and subjected to column chromatography eluting with 3:1 ethyl acetate in hexanes to remove the unreacted aldehyde, followed by eluting with 1:1 ethyl acetate in hexanes, yielding the desired product (Compound 25, cyclohexyl, cis/trans mixture). The solvents are removed in vacuo (typical general yields range from 50 to 80%).

Preparation of the Acylated Derivative Followed by Deprotection of Guanidine:

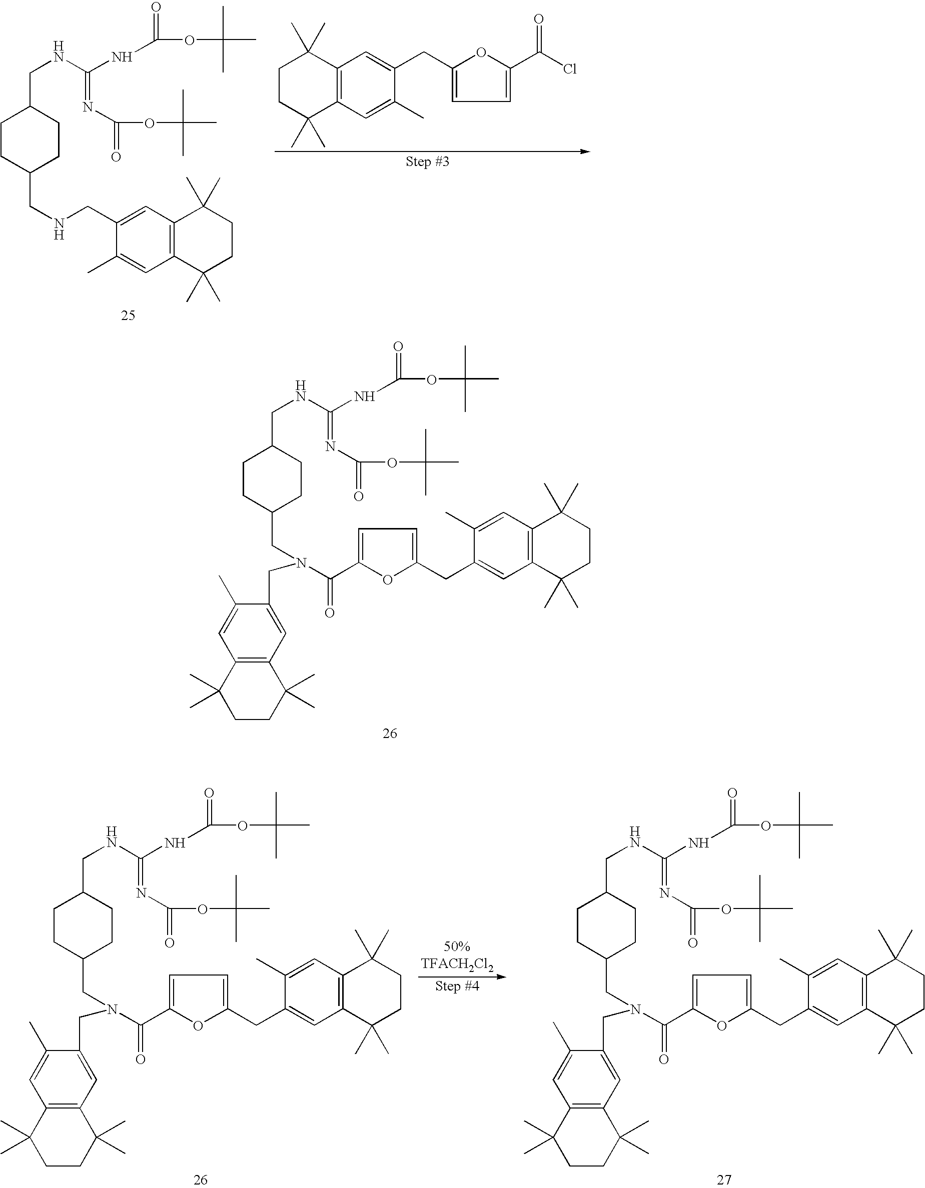

The product from the reductive amination 25 (1.0 equiv.) is dissolved in dichloromethane (10-15 mL), followed by the addition of triethylamine (2 equiv.), and 2-furoyl chloride reagent (1.0 equiv.). The reaction contents are stirred overnight at room temperature. The reaction is diluted with dichloromethane (50 mL) and washed with saturated sodium bicarbonate. The organic layer is dried over magnesium sulfate, filtered, and purified by column chromatography and eluted using 3:1 hexanes in ethyl acetate. The solvents are removed in vacuo to give Compound 26.

The product from the acylation reaction 26 (1.0 equiv.) is dissolved in a solution of 50% TFA in dichloromethane (20-25 mL), and the reaction contents are stirred at room temperature (15-20 minutes; solution becomes slight reddish-orange). The reaction contents are stirred for an additional 1 hour and 20 minutes until the deprotection is complete. The reaction is terminated by concentration in vacuo, followed by the addition of water/acetonitrile (˜50 mL) and lyophilization overnight. The final compound is purified by HPLC methods. The solvents are removed in vacuo to give Compound 27.

The following discussion relates to the preparation of exemplary Compounds (e)-(k). Compounds (e)-(k) may be used as described above to produce the corresponding deprotected (free guanidinyl) compounds, through hydrolysis under acid conditions.

Preparation of 1-(N,N′-diBoc)-guanidinomethyl-3-aminomethylcyclohexane:

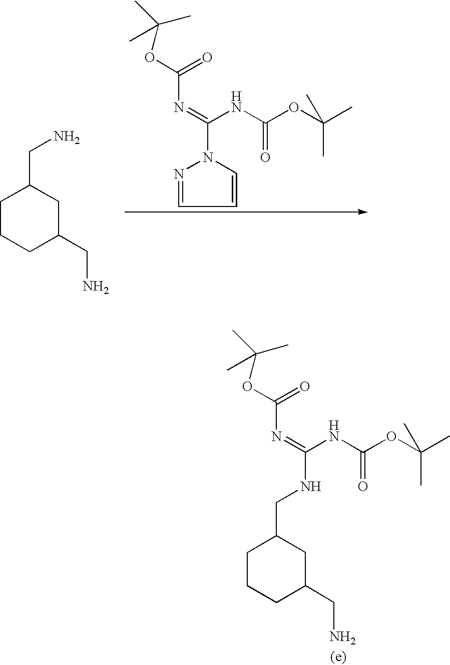

To a solution of cis/trans-1,3-bis-aminomethylcyclohexane (7.5 g, 52.8 mmol) in THF (30 mL) is added a solution of 1,3-bis(tert-butoxycarbonyl)-2-methyl-2-thiopseudourea (7.65 g, 26.3 mmol) in THF (40 mL) within 0.5 hour. The solution is stirred at room temperature for 5 hours. The solvent is removed under reduced pressure, and the product is purified by column chromatography on silica gel using a mixture of methylene chloride/methanol as the eluant, to afford 2.2 g (22% yield) of 1-(N,N′-diBoc)-guanidinomethyl-3-aminomethylcyclohexane (Compound (e)). ¹H NMR (CDCl₃) 67 11.53 (br s, 1H), 8.40 (br s, 1H), 3.28-3.30 (m, 2H), 2.54-2.61 (m, 2H), 1.81 (br s, 2H), 1.27-1.58 (m, 26H), 0.89 (m, 1H), 0.65 (m, 1H).

Alternatively, Compound (e) may be prepared as follows. To a solution of cis/trans 1,3-bis-aminomethylcyclohexane (10.0 g, 70.3 mmol) in THF (1000 mL, 0.07M) is added portionwise as a solid (over a 10-minute period) 1-H-Pyrazole-1-(N,N-bis(tert-butoxycarbonyl)carboxamidine) (21.8 g, 70.3 mmol). The solution is stirred at room temperature for 0.5 hour. The solvent is removed under reduced pressure to give a syrupy residue, which is taken up in ethyl acetate (500 mL) and washed twice with water. The layers are separated, and the product is purified by column chromatography on silica gel and eluted with 100% ethyl acetate to remove any non-polar impurities, followed by elution with 100% isopropyl alcohol to give the pure product. The solvents are removed in vacuo to afford 11.4 g (41% yield) of 1-(N,N′-diBoc)-guanidinomethyl-3-aminomethylcyclohexane. ¹H NMR (CDCl₃) δ 11.53 (br s, 1H), 8.40 (br s, 1H), 3.28-3.30 (m, 2H), 2.54-2.61 (m, 2H), 1.81 (br s, 2H), 1.27-1.58 (m, 26H), 0.89 (m, 1H), 0.65 (m, 1H).

Preparation of 1-(N,N′-diBoc)-guanidinomethyl-4-aminomethylbenzene:

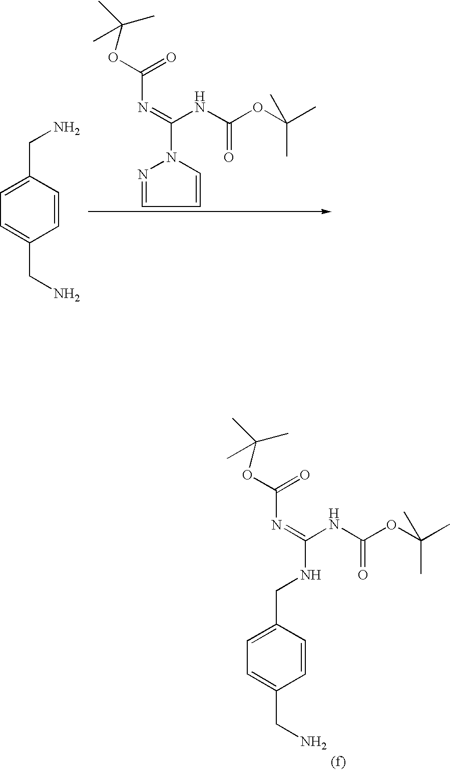

To a solution of p-xylylenediamine (6.44 g, 47.4 mmol) in THF (30 mL) is added a solution of 1,3-bis(tert-butoxycarbonyl)-2-methyl-2-thiopseudourea (6.63 g, 22.9 mmol) in THF (40 mL) within 0.5 hour. The solution is stirred at room temperature for 5 hours. The solvent is removed under reduced pressure, and the product is purified by column chromatography on silica gel using a mixture of methylene chloride/methanol as the eluant, to afford 8.0 g (92% yield) of 1-(N,N′-diBoc)-guanidinomethyl-4-aminomethyl benzene (Compound (f)). ¹H NMR (CDCl₃) δ 11.54 (br s, 1H), 8.56 (br s, 1H), 7.29 (s, 4H), 4.60 (d, 2H), 3.86 (s, 2H), 1.64 (br s, 2H), 1.52 (s, 9H), 1.48 (s, 9H).

Preparation of 1-(N,N′-diBoc)-guanidinomethyl-3-aminomethylbenzene:

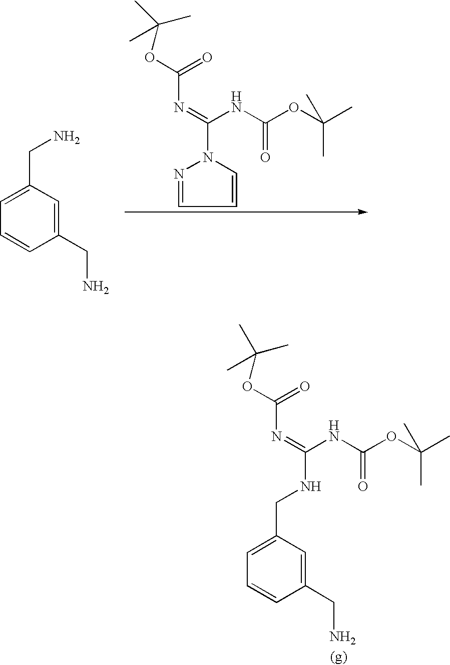

To a solution of m-xylylenediamine (7.14 g, 52.5 mmol) in THF (30 mL) is added a solution of 1,3-bis(tert-butoxycarbonyl)-2-methyl-2-thiopseudourea (7.57 g, 26.1 mmol) in THF (40 mL) within 0.5 hour. The solution is stirred at room temperature for 5 hours. The solvent is removed under reduced pressure, and the product is purified by column chromatography on silica gel using a mixture of methylene chloride/methanol as the eluant, to afford 7.9 g (80% yield) of 1-(N,N′-diBoc)-guanidinomethyl-3-aminomethylbenzene (Compound (g)). ¹H NMR (CDCl₃) δ 11.54 (br s, 1H), 8.58 (br s, 1H), 7.19-7.34 (m, 4H), 4.62 (d, 2H), 3.86 (s, 2H), 1.83 (br s, 2H), 1.52 (s, 9H), 1.48 (s, 9H).

Preparation of 1-(N,N′-diBoc)-guanidine-4-aminobutane:

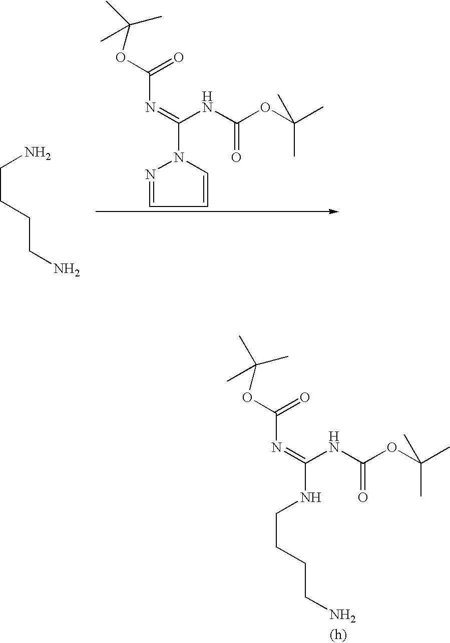

To a solution of 1,4-diaminobutane (4.15 g, 47.1 mmol) in THF (30 mL) is added a solution of 1,3-bis(tert-butoxycarbonyl)-2-methyl-2-thiopseudourea (6.83 g, 23.6 mmol) in THF (40 mL) within 0.5 hour. The solution is stirred at room temperature for 5 hours. The solvent is removed under reduced pressure, and the product is purified by column chromatography on silica gel using a mixture of methylene chloride/methanol as the eluant, to afford 3.0 g (40% yield) of 1-(N,N′-diBoc)-guanidino-4-aminobutane (Compound (h)). ¹H NMR (CDCl₃) δ 11.49 (br s, 1H), 8.35 (br s, 1H), 3.42-3.47 (m, 2H), 2.72-2.76 (t, 2H), 0.86-1.65 (m, 24H).

An alternate procedure for preparing Compound (h) is as follows. To a solution of 1,4-diaminobutane (6.0 g, 68.1 mmol) in THF (972 mL, 0.07M) is added portionwise as a solid (over a 10-minute period) 1-H-pyrazole-1-(N,N-bis(tert-butoxycaarbonyl)carboxamidine) (21.5 g, 68.1 mmol). The solution is stirred at room temperature for 0.5 hour. The solvent is removed under reduced pressure to give a syrupy residue, which is taken up in ethyl acetate (500 mL) and washed twice with water. The layers are separated and the product is purified by column chromatography on silica gel and eluted with 100% ethyl acetate to remove any non-polar impurities and then with 100% isopropyl alcohol to give the pure product. The solvents are removed in vacuo to afford 10.0 g (44% yield) of 1-(N,N′-diBoc)-guanidino-4-aminobutane. ¹H NMR (CDCl₃) δ 11.49 (br s, 1H), 8.35 (br s, 1H), 3.42-3.47 (m, 2H), 2.72-2.76 (t, 2H), 0.86-1.65 (m, 24H).

Preparation of 1-N,N-dimethylaminomethyl-4-aminomethylbenzene:

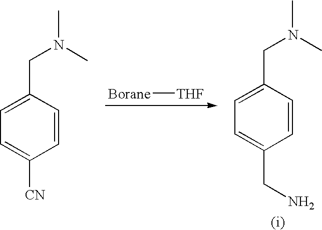

To a solution of 1-N,N-dimethyl aminomethyl-4-carbonitrile benzene (4.8 g, 30 mmol) in THF is added a solution of 1 M borane tetrahydrofuran complex (90 mL). The mixture is heated at reflux temperature for 16 hours under nitrogen. After cooling to room temperature, a 1M solution of HCl in methanol (100 mL) is added. The reaction mixture is heated at reflux for 3 hours. The product, which precipitates, is collected by filtration, washed with diethyl ether, and dried in vacuo to give 5.9 g (83% yield) of the product as the hydrochloride salt (Compound (i)): ¹H NMR (DMSO-d₆) δ 8.65 (br s, 3H), 7.55 (dd, 4H), 4.25 (s, 2H), 3.98 (s, 2H), 2.62 (s, 6H).

Preparation of 1-(N,N′-diBoc)-guanidinomethyl-2-aminomethylbenzene:

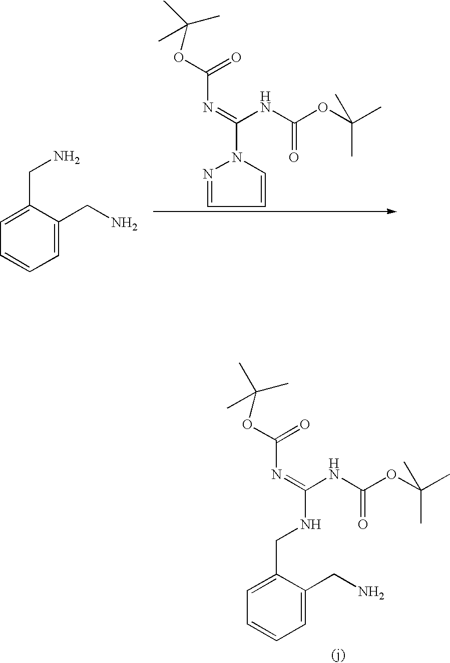

To a solution of o-xylylenediamine (7.14 g, 52.5 mmol) in THF (30 mL) is added a solution of 1,3-bis(tert-butoxycarbonyl)-2-methyl-2-thiopseudourea (7.57 g, 26.1 mmol) in THF (40 mL) within 0.5 hour. The solution is stirred at room temperature for 5 hours. The solvent is removed under reduced pressure, and the product is purified by column chromatography on silica gel using a mixture of methylene chloride/methanol as the eluant, to afford 1-(N,N′-diBoc)-guanidinomethyl-3-aminomethyl benzene (Compound (j)).

Alternatively, Compound (j) may be prepared in a manner analogous to the alternative preparation described above for Compound (e).

Preparation of 1-(N,N′-diBoc)-guanidinomethyl-2-aminomethylcyclohexane:

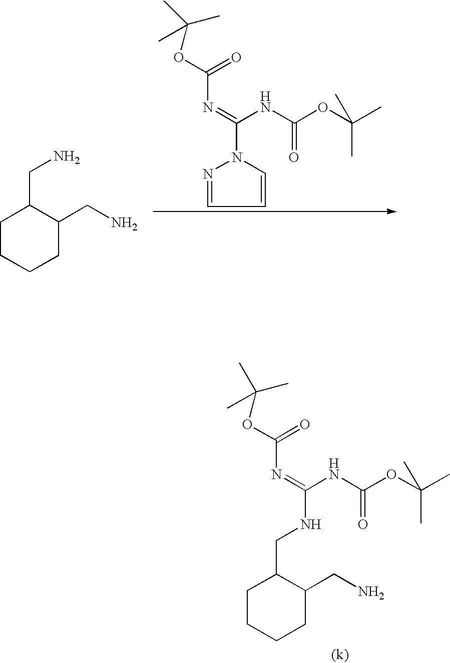

To a solution of cis/trans-1,2-bis-aminomethylcyclohexane (7.5 g, 52.8 mmol) in THF (30 mL) is added a solution of 1,3-bis(tert-butoxycarbonyl)-2-methyl-2-thiopseudourea (7.65 g, 26.3 mmol) in THF (40 mL) within 0.5 hour. The solution is stirred at room temperature for 5 hours. The solvent is removed under reduced pressure, and the product is purified by column chromatography on silica gel using a mixture of methylene chloride/methanol as the eluant, to afford 1-(N,N′-diBoc)-guanidinomethyl-2-aminomethylcyclohexane (Compound (k)).

Alternatively, Compound (k) may be prepared in a manner analogous to the alternative preparation described above for Compound (e).

D. Pyrimidine Compounds

Pyrimidines can be utilized according to the following procedures:

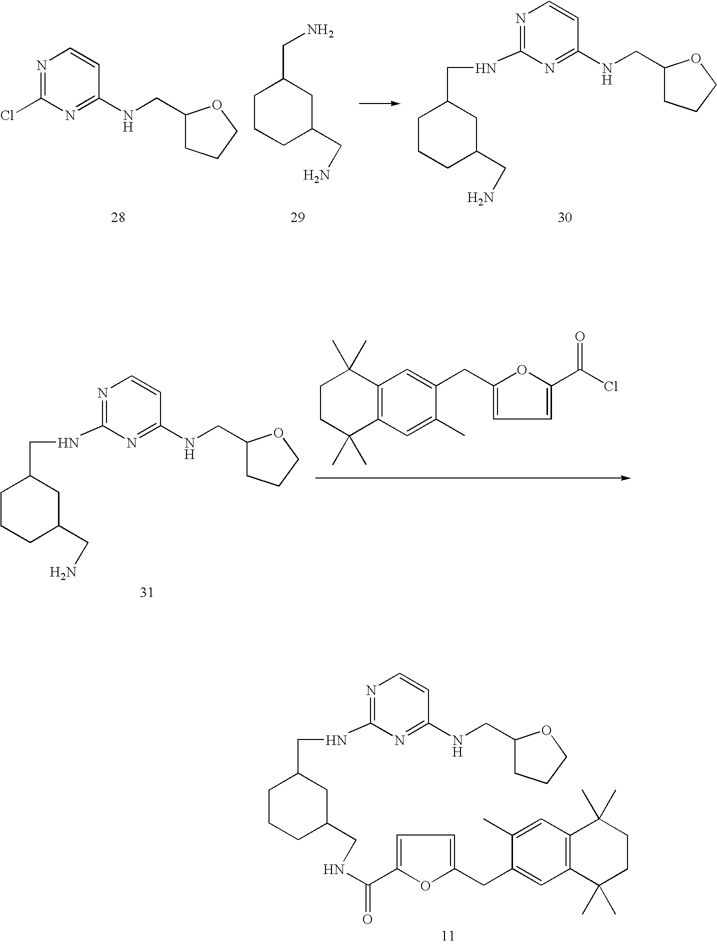

A general procedure for the preparation of pyrimidine containing compounds is as follows. To a solution of 1,3 diamine 29 in THF is added 28 and the contents refluxed for 12 hours. The solvents are removed in vacuo and the desired adduct purified by column chromatography. Pure 31 is acylated according to the general procedure given above to give 11.

As skilled artisans will appreciate, a variety of compounds according to the invention may be prepared based on the above teachings. The chemical reactions described above have general applicability to the preparation of the GnRH agents of the invention. Thus, other GnRH agents may be similarly prepared by suitable modification as will be readily appreciated by those skilled in the art, e.g., by protection of interfering groups, by adapting for use with other conventional reagents, and/or by routine modifications of reaction conditions.

### IN VITRO PHARMACOLOGY RADIOLIGAND BINDING

Cell membranes prepared from human embryonic kidney 293 cells stably transfected with cDNA for the human GnRH receptor were suspended in binding assay buffer containing: 50 mM HEPES, 1 mM EDTA, 2.5 mM MgCl₂, and 0.1% bovine serum albumin. Membranes (5-50 μg total protein per well containing approximately 10-100 fmol of GnRH receptor) were incubated in duplicate in 96-well plates in 200 μl total volume with ¹²⁵I-GnRH-A (approximately 0.05 nM) and test compounds for one hour at room temperature. All compounds were diluted in 1% DMSO (final assay concentration) in binding assay buffer. Nonspecific binding was determined in the presence of 100 nM GnRH. Reactions were terminated by rapid filtration onto 96-well Packard GF/C filters soaked in 0.1% polyethyleneimine. Filters were washed three times with PBS buffer, dried and counted on a Packard Topcount by liquid scintillation counting.

Assay conditions were identical for assessing compound activities at other species. A similar number of GnRH receptors was utilized for each species assay. For rat GnRH receptor binding, membranes were prepared from rat pituitary and approximately 25-30 μg/well of total membrane protein were utilized. For bovine GnRH receptor binding, membranes were prepared from bovine pituitary and utilized at 40-50 μg/well. For mouse GnRH receptor binding, membranes were prepared from 293 cells stably expressing mouse GnRH receptors and were utilized at approximately 25-30 μg/well. IC₅₀ values for control peptides and test compounds were calculated utilizing GraphPad Prism™ software. The result of a radioligand binding experiment is shown in FIG. 1. Table 1 shows mean values from multiple experiments of the affinities of various peptide and non-peptide compounds at GnRH receptors from four species.

[image]

FIG. 1. Effects of compounds on ¹²⁵I-GnRH-A binding to hGnRH receptors expressed in HEK-293 cells. The ability of GnRH (squares) and 9 (triangles) to displace ¹²⁵I-GnRH-A (approximately 0.05 nM) binding to hGnRH receptors was examined. Values shown are from one representative experiment performed in duplicate.

Various compounds of the Formula I were synthesized according to the general reaction scheme generally described above. Crude compounds were tested using the competitive radioligand binding assay described above. Results of the GnRH competitive binding assay are shown in the table (each compound tested at 1 or 10 μM).

TABLE 1

| | Human IC₅₀ | Bovine IC₅₀ | Rat IC₅₀ | Mouse IC₅₀ |
|---|---|---|---|---|
| Compound | (nM) | (nM) | (nM) | (nM) |
| GnRH | 7.2 ± 1.5 | 13 ± 2 | 33 ± 1.9 | 11 ± 2 |
| GnRH-A | 0.34 ± 0.06 | 0.3 ± 0.05 | 0.49 ± 0.1 | 0.22 ± 0.03 |
| Antide | 0.67 ± 0.09 | 0.15 ± 0.02 | 0.19 ± 0.04 | 0.25 ± 0.05 |
| 9 | 220 ± 33 | 3800 ± 220 | 680 ± 120 | 2300 ± 460 |
| 10 | 130 ± 24 | 1500 ± 480 | 390 ± 10 | 1400 ± 440 |
| 11 | 190 ± 40 | 320 ± 10 | 9.0 ± 0.3 | 50 ± 10 |
| 12 | 230 ± 37 | 10400 ± 3000 | 3080 ± 630 | 7130 ± 1350 |
| 13 | 110 ± 20 | 530 ± 100 | 60 ± 8 | 120 ± 20 |
| 14 | 80 ± 4 | 1050 ± 30 | 60 ± 15 | 290 ± 70 |
| 15 | 100 ± 17 | 1000 ± 240 | 70 ± 16 | 220 ± 50 |
| 16 | 30 ± 6 | 4380 ± 510 | 560 ± 50 | 1290 ± 210 |
| 17 | 80 ± 20 | 670 ± 120 | 30 ± 4 | 80 ± 20 |
| 18 | 55 ± 11 | 460 ± 90 | 40 ± 3 | 115 ± 25 |
| 19 | 50 ± 3 | ND | ND | ND |
| 20 | 8.0 ± 0.9 | ND | ND | ND |

Values are means ±SE of at least three experiments performed in duplicate.
ND = not determined.

### TOTAL INOSITOL PHOSPHATES MEASUREMENT

To assess the activity of the compounds as agonists or antagonists, an assay measuring accumulation of total inositol phosphates was employed. 293 cells containing the hGnRH receptor were plated onto 24-well plates (approximately 200,000 cells/well) using DMEM media. The following day, cells were loaded with [³H]myoinositol (0.5 Ci/ml) for 16-18 hours in inositol-free medium. The medium was aspirated and the cells rinsed with serum-free DMEM. The medium was aspirated and the cells were then treated with test compounds or vehicle for 30 minutes at 37° C. A half-maximal concentration of GnRH (1 nM) or vehicle was then added to the cells and allowed to equilibrate at 37° C. for 45 minutes. The media was replaced with ice-cold 10 mM formic acid, which stopped the reaction and also served to extract cellular lipids. Inositol phosphates were separated by ion-exchange chromatography on Dowex columns, which were washed with 2.5 mL of 10 mM myoinositol and 10 mM formic acid. The columns were then washed with 5 mL of 60 mM sodium formate and 5 mM borax, and total inositol phosphates were eluted with 5 mL 1M ammonium formate, 0.1 M formic acid. The column eluates were added to liquid scintillation vials containing 15 ml of scintillation cocktail and were counted by liquid scintillation counting. The result of a typical experiment is shown in FIG. 2.

[image]

FIG. 2. Effects of compounds on GnRH-stimulated total inositol phosphate accumulation in HEK-293 cells expressing the hGnRH receptor. The ability of the peptide antagonist, Antide, and non-peptide compound 9 to block GnRH-stimulated increases in [³H]inositol phosphates was examined. Neither compound alone stimulated an increase in total [³H]inositol phosphates (not shown), but both compounds were able to inhibit the stimulation mediated by a half-maximal concentration of GnRH peptide. GnRH alone dose-dependently increased [³H]inositol phosphate accumulation with an EC₅₀ of approximately 0.8 nM. In the experiment shown, the K_{b} values of Antide and compound 9 were determined by the method of Cheng and Prusoff (Biochem. Pharmacol. 22:3099-3108, 1973). Values shown are from one experiment performed in duplicate.

### IN VIVO PHARMACOLOGY ANIMAL EFFICACY STUDIES

Experimental Protocol: Male Sprague-Dawley (225-250 g) rats were castrated and allowed 10 days post-operative recovery. Ten days post castration animals were instrumented with indwelling femoral venous and arterial catheters to facilitate remote infusions and blood sampling. On the day of the experiment, animals were allowed to acclimate to the procedure room while residing in their home cage. Basal blood samples were drawn from all animals. Following basal sampling, either vehicle (10% DMSO, 10% cremophor/saline), Antide (1.0 μg) or compound 11 (10 mg/kg) was administered intravenously. Blood samples were drawn 10, 60, 90, 120, 180, 240 minutes after injections. Blood was centrifuged, serum collected and stored in −70° freezer until assayed. Serum samples were analyzed using DSL-4600 ACTIVE LH coated-tube immunoradiometric assay kit from Diagnostic Systems Laboratories, Inc.

Results and discussion: Removal of the gonads eliminates the negative feedback of testosterone on the hypothalamus, resulting in elevated GnRH and consequently elevated LH. FIG. 3 illustrates the plasma levels of both LH and testosterone in control and castrated rats 10 days after surgery. In these rats, a GnRH antagonist would be expected to reduce GnRH mediated elevations of LH levels. Antide, a peptide GnRH antagonist, reduces LH in the castrated rat model (FIG. 4). Compound 11, a small-molecule GnRH antagonist, also suppresses LH in the castrated rat model (FIG. 4).

[image]

[image]

### PHARMACOKINETIC STUDIES

Experimental protocol: Rats were prepared with intravenous catheters inserted in the superior vena cava through the incision in the right external jugular vein and allowed to recover. Drugs were dissolved in a mixture of 10% DMSO, 10% cremaphor, and 80% saline and administered i.v. at a dose of 10 mg/kg. Blood samples were taken at the times indicated, and the compounds were extracted from 0.2 mL of plasma with butyl chloride containing an internal standard. Samples were analyzed by HPLC on a Beta-Basic C18 4×50 mm column using a gradient of 40-80% acetonitrile in 10 mM ammonium phosphate buffer at a flow rate of 1 ml/min. Sample detection was by UV absorbance at 260 nm.

Results and Discussion: Compound 11, which has excellent affinity at the rat GnRH receptor, had a half life in rat plasma of approximately three hours and had a concentration in plasma of 100-200 nM four hours after i.v. injection (FIG. 5).

[image]

Binding of the reference peptides to rat, mouse, bovine and human GnRH receptors are in good agreement with those reported in the literature. Non-peptide compounds of the invention show marked species differences in their binding profile. Several of these compounds exhibit high affinity (<100 nM) at the human GnRH receptor. Functionally, all of these non-peptide compounds assessed for activity in an inositol phosphate assay act as antagonists of GnRH-stimulated total inositol phosphate accumulation in cells containing recombinant human GnRH receptors. Intravenous administration of compound 11 reduced plasma levels of LH in castrated male rats, a model for chronically elevated plasma LH levels. This compound has a half life of three hours, and the plasma concentration correlated with efficacy. Taken together, these data suggest that these non-peptide compounds should have utility as GnRH receptor antagonists.

Peptide Agonists and Antagonists Used as Reference Compounds:

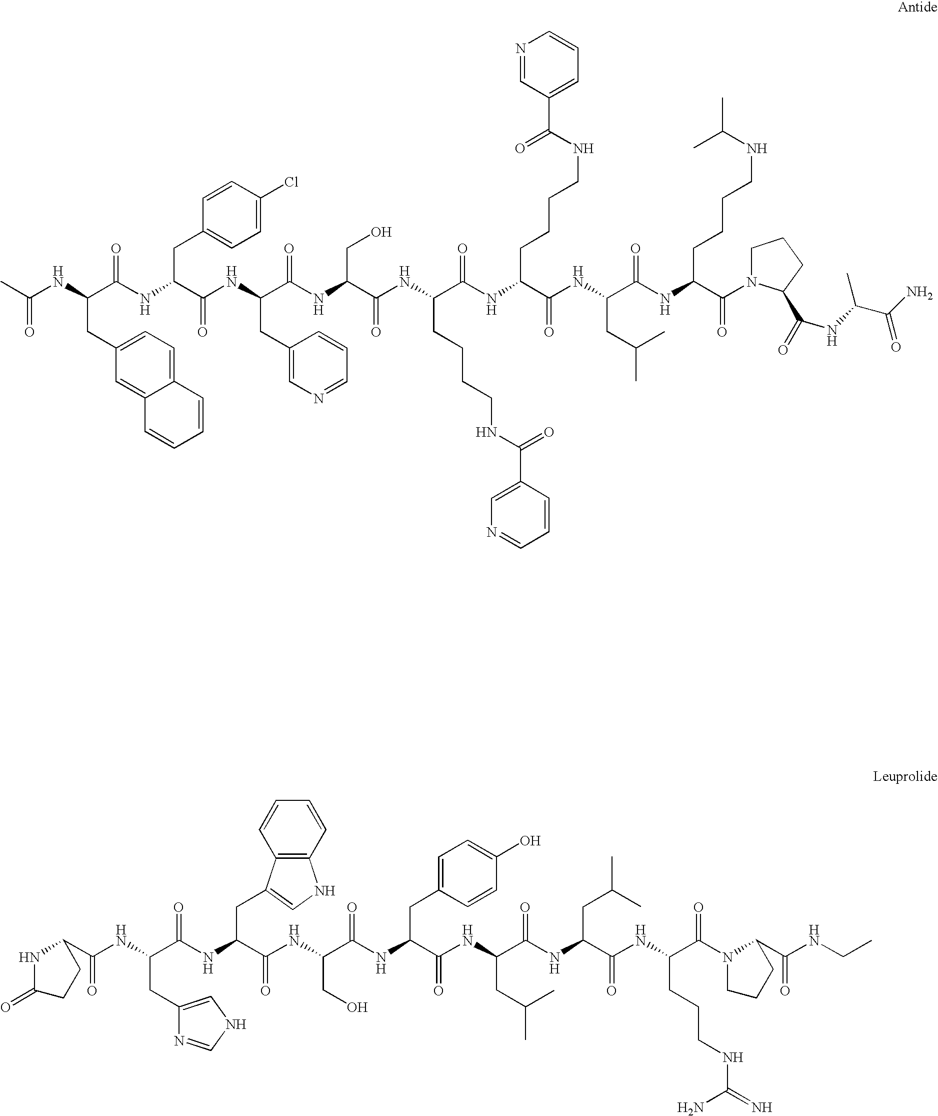

The invention has been illustrated by reference to preferred embodiments and exemplary aspects of the invention. Various modifications and adaptations will become apparent to the artisan through routine practice of the invention in light of knowledge and developments in the art. Thus, the invention should be understood as not being limited by the foregoing detailed description, but as being defined by the appended claims and their equivalents.

What is claimed is:

1. A compound having a formula selected from the group consisting of: 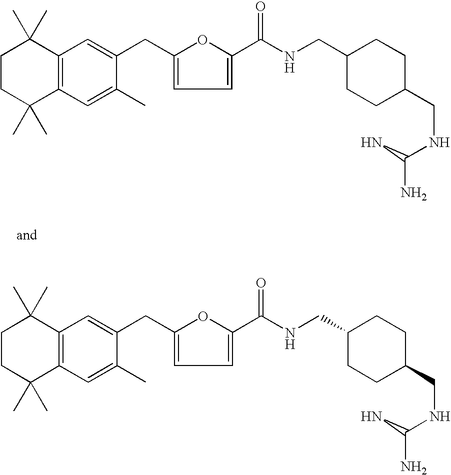
or a pharmaceutically acceptable salt, multimer, prodrug, or active metabolite thereof.

2. A compound having a formula selected from the group consisting of: 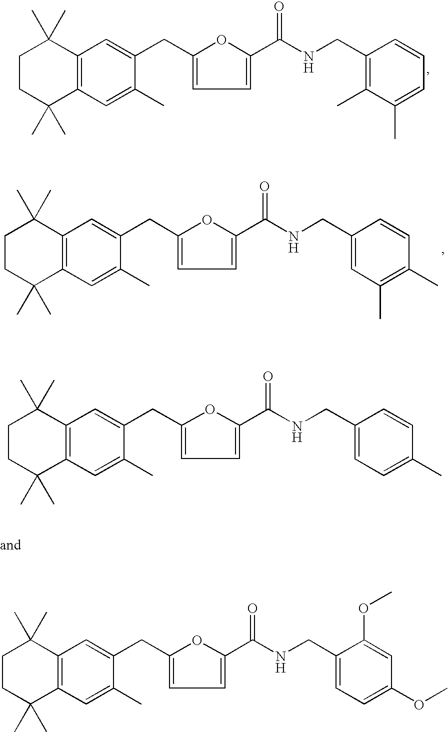
or a pharmaceutically acceptable salt, multimer, prodrug, or active metabolite thereof.

3. A compound having the formula: 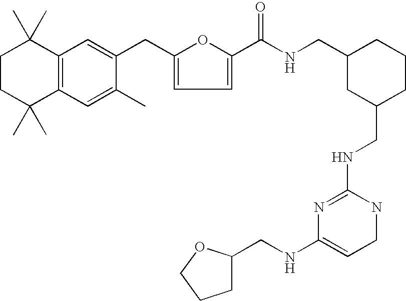
or a pharmaceutically acceptable salt, multimer, prodrug, or active metabolite thereof.

4. A compound having a formula selected from the group consisting of: 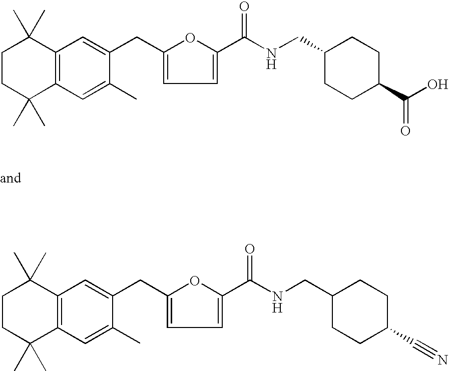
or a pharmaceutically acceptable salt, multimer, prodrug, or active metabolite thereof.

5. A compound having the formula: 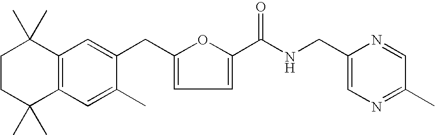
or a pharmaceutically acceptable salt, multimer, prodrug, or active metabolite thereof.

6. A compound having a formula selected from the group consisting of: 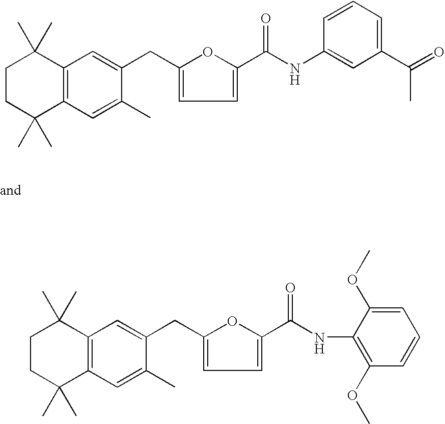
or a pharmaceutically acceptable salt, multimer, prodrug, or active metabolite thereof.

7. A pharmaceutical composition comprising: a therapeutically effective amount of a compound, pharmaceutically acceptable salt, multimer, prodrug, or active metabolite as defined in any of claims 1-6; and a pharmaceutically acceptable carrier or diluent.

8. A method for regulating the secretion of gonadotropins in mammals, comprising administering a therapeutically effective amount of a compound, pharmaceutically acceptable salt, multimer, prodrug, or active metabolite as defined in any of claims 1-6.
